(19) United States
(12) Patent Application Publication
(10) Pub. No.: US 2025/0204875 A1
(43) Pub. Date: Jun. 26, 2025

(54) UNIVERSAL ADAPTOR FOR AN IMAGING DEVICE AND CORRECTOR ASSEMBLY

(71) Applicant: Mazor Robotics Ltd., Caesarea (IL)

(72) Inventors: Yinan Liu, Shanghai (CN); Zhinan Guo, Shanghai (CN); Wei Tang, Shanghai (CN); Jiangfeng Zhang, Shanghai (CN); Siyao Zhu, Shanghai (CN); Ying Chen, Shanghai (CN)

(21) Appl. No.: 18/850,136

(22) PCT Filed: Mar. 25, 2022
(86) PCT No.: PCT/CN2022/082933

§ 371 Date: Sep. 24, 2024

(51) Int. Cl. A61B 6/00

(52) U.S. Cl. CPC A61B 6/4411

(57) ABSTRACT

An adaptor assembly (300, 700, 1100, 1800) comprising a plurality of spacers (722, 724, 726, 728, 1126, 1128, 1130) configured to contact a plurality of contact points of an imaging device (100) and a plurality of holders (120) configured to be adjustable in one or more directions and to support the plurality of spacers (722, 724, 726, 728, 1126, 1128, 1130). The adaptor assembly (300, 700, 1100, 1800) is configured to couple a corrector assembly (104) to an imaging device (100). The plurality of holders (120) may comprise a bracket (702) and at least one side bracket (704); a plurality of arms (304, 306, 308), each arm comprising a first link (314) pivotably connected to a base (312) and a second link (316) pivotably connected to the first link (314); at least one clamp assembly (1102, 1104, 1106); or at least one belt assembly (1802, 1804, 1806).

### FIELD

The present technology generally relates to coupling a corrector assembly to an imaging device, and relates more particularly to an adaptor for coupling the corrector assembly to the imaging device.

### BACKGROUND

Image devices may be used by a medical provider to obtain images for diagnostic and/or therapeutic purposes. Images obtained from the imaging device may include distortions or blurriness. Such defects may be corrected or a quality of the image may be improved by hardware attached to the image devices.

### SUMMARY

Example aspects of the present disclosure include:

An adaptor assembly according to at least one embodiment of the present disclosure comprises a first arm, a second arm, and a third arm each comprising a first link pivotably connected to a base and a second link pivotably connected to the first link; a clamping bar supported by the first arm and pivotably connected to the first link of the first arm; a first side bar supported by the second arm and pivotably connected to the first link of the second arm; and a second side bar supported by the third arm and pivotably connected to the first link of the second arm, wherein the clamping bar, the first side bar, and the second side bar are securable to an imaging device.

Any of the aspects herein, further comprising a first stopper configured to lock a corresponding first link and a second stopper configured to lock a corresponding second link.

Any of the aspects herein, further comprising a grip disposed on each of the clamping bar, the first side bar, and the second side bar.

Any of the aspects herein, wherein the first side bar and the second side bar are each coupled to the clamping bar via a corresponding strap, wherein each strap is adjustable by a corresponding adjuster.

Any of the aspects herein, wherein the clamping bar, the first side bar, and the second side bar contact an imaging device at a plurality of contact points, and wherein each strap is adjusted to tighten and clamp each of the clamping bar, the first side bar, and the second side bar to the imaging device.

An adaptor assembly according to at least one embodiment of the present disclosure comprises a bracket comprising a wall extending perpendicularly from a base, a spacer laterally adjustable on the wall, and an adjustor configured to pivotably adjust the spacer; and at least one side bracket, each side bracket comprising a wall extending perpendicularly from a base and a spacer laterally adjustable on the wall, wherein the spacer of the bracket and each spacer of the at least one side bracket are configured to contact an imaging device at a plurality of contact points.

Any of the aspects herein, wherein the wall of each of the at least one side bracket comprises a plurality of apertures spaced along a length of the wall, wherein the plurality of apertures are configured to receive the spacer, wherein the spacer is laterally adjustable by removing the spacer from a first set of apertures and securing the spacer to a second set of apertures.

Any of the aspects herein, wherein the adjustor comprises a trigger and a protrusion each coupled to an adjustor base, wherein the protrusion is received in at least one of a first aperture and a second aperture spaced from the first aperture, wherein actuation of the trigger moves the protrusion from at least one of the first aperture and the second aperture, and wherein the adjustor base is pivotable when the trigger is actuated.

Any of the aspects herein, wherein the trigger is biased away from the adjustor base.

Any of the aspects herein, wherein the at least one side bracket comprises a first side bracket, a second side bracket, and a third side bracket.

An adaptor assembly according to at least one embodiment of the present disclosure comprises a first clamp assembly; a second clamp assembly; and a third clamp assembly, wherein each of the first clamp assembly, the second clamp assembly, and the third clamp assembly comprises a clamp bracket extending from a clamp base, a clamp arm supported by the clamp bracket, and a spacer supported by the clamp arm, the spacer adjustable laterally, longitudinally, and rotationally.

Any of the aspects herein, wherein the first clamp assembly also comprises an adjustor for pivotably adjusting the spacer.

Any of the aspects herein, wherein the clamp arm of each assembly comprises a slot through which a threaded portion of the spacer extends through, wherein the spacer is slidable along the slot and securable to the clamp arm by a nut threading to the threaded portion.

Any of the aspects herein, wherein each assembly comprises a lateral adjustor comprising a knob and a threaded shaft extending from the knob, wherein the clamp arm includes a threaded aperture configured to receive the threaded shaft, and wherein rotation of the knob in a first direction moves the clamp arm in a first lateral direction and rotation of the knob in a second direction moves the clamp arm in a second lateral direction opposite the first lateral direction.

Any of the aspects herein, wherein the spacer of each assembly comprises a shaft and a contact point coupled to an end of the shaft in a ball and joint configuration.

Any of the aspects herein, wherein each assembly comprises a lateral adjustor comprising an aperture disposed on the clamp arm, wherein the aperture comprises an inner surface having a threaded bore and a non-threaded bore, wherein an axis of the threaded bore is perpendicular to a center line of the clamp arm and an axis of the non-threaded bore is oblique to the center line.

Any of the aspects herein, wherein the lateral adjustor further comprises a threaded shaft extending through the aperture, wherein when the clamp arm is in a first position, the threaded bore is engaged with the threaded shaft and when the clamp arm is in a second position, the threaded bore is disposed in the non-threaded bore.

An adaptor assembly according to at least one embodiment of the present disclosure comprises a first belt assembly; a second belt assembly; and a third belt assembly, wherein each of the first belt assembly, the second belt assembly, and the third belt assembly comprises a first belt coupled to a belt base at one end and a ratchet at another end, a second belt coupled to the ratchet and a contact point of a plurality of contact points, wherein the ratchet is configured to tighten the second belt, wherein the second belt of each of the first belt assembly, second belt assembly, and the third belt assembly attach to a first contact point, a second contact point, and a third contact point, wherein the first contact point is opposite the second contact point and the third contact point.

Any of the aspects herein, wherein the plurality of contact points comprises a plurality of handle rails.

Any of the aspects herein, wherein the belt base of each assembly comprises a plate and at least one belt arm, wherein the plate is configured to secure the assembly to a corrector assembly and the at least one belt arm configured to receive the first belt.

Any of the aspects herein, wherein the ratchet comprises a central gear configured to engage with a first side gear and a second side gear, wherein the first side gear and the second side gear are configured to rotate the central gear when the first side gear and the second side gear are rotating and are engaged with the central gear.

Any of the aspects herein, wherein the first side gear and the second side gear are configured to disengage the central gear when a force applied to the first side gear and the second side gear exceed a threshold force.

Any of the aspects herein, wherein the first side gear and the second side gear are biased to the central gear by a biasing force, and wherein the first side gear and the second side gear are configured to disengage the central gear when the force applied is greater than the biasing force and the threshold force.

Any of the aspects herein, wherein each of the first side gear, the second side gear, and the central gear comprise a crown gear.

Any of the aspects herein, wherein the ratchet comprises at least one spring configured to apply the biasing force.

An adaptor assembly according to at least one embodiment of the present disclosure comprises a plurality of spacers configured to contact a plurality of contact points; and a plurality of holders configured to be adjustable in one or more directions and to support the plurality of spacers.

Any of the aspects herein, wherein the plurality of holders comprises a bracket comprising a wall extending perpendicularly from a base, one spacer of the plurality of spacers laterally adjustable on the wall, and an adjustor configured to pivotably adjust the spacer, and wherein the plurality of holders further comprises at least one side bracket, each side bracket comprising a wall extending perpendicularly from a base and a corresponding spacer of the plurality of spacers laterally adjustable on the wall.

Any of the aspects herein, further comprising a first arm, a second arm, and a third arm, each comprising a first link pivotably connected to a base and a second link pivotably connected to the first link; and wherein the plurality of holders comprises a clamping bar supported by the first arm and pivotably connected to the first link of the first arm, a first side bar supported by the second arm and pivotably connected to the first link of the second arm, and a second side bar supported by the third arm and pivotably connected to the first link of the second arm.

Any of the aspects herein, wherein the plurality of holders comprises a first clamp assembly; a second clamp assembly; and a third clamp assembly, wherein each of the first clamp assembly, the second clamp assembly, and the third clamp assembly comprises a clamp bracket extending from a clamp base, a clamp arm supported by the clamp bracket, and a spacer supported by the clamp arm, the spacer adjustable laterally, longitudinally, and rotationally.

Any of the aspects herein, wherein the plurality of holders comprises a first belt assembly, a second belt assembly, and a third belt assembly, wherein each of the first belt assembly, the second belt assembly, and the third belt assembly comprises a first belt coupled to a belt base at one end and a ratchet at another end, a second belt coupled to the ratchet and a contact point of a plurality of contact points, and wherein the ratchet is configured to tighten the second belt.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present invention will become apparent to those skilled in the art upon consideration of the embodiment descriptions provided hereinbelow.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1A is a perspective view of an imaging device and a corrector assembly according to at least one embodiment of the present disclosure;

FIG. 1B is a side view of an imaging device and a corrector assembly according to at least one embodiment of the present disclosure;

FIG. 2A is a top view of a corrector assembly according to at least one embodiment of the present disclosure;

FIG. 2B is a perspective view of a corrector assembly according to at least one embodiment of the present disclosure;

FIG. 3 is a perspective view of an adaptor according to at least one embodiment of the present disclosure;

FIG. 4 is a perspective view of an adaptor and a corrector assembly according to at least one embodiment of the present disclosure;

FIG. 5A is a side view of an adaptor and a corrector assembly according to at least one embodiment of the present disclosure;

FIG. 5B is a top view of an adaptor and a corrector assembly according to at least one embodiment of the present disclosure;

FIG. 6A is a perspective view of an adaptor and a corrector assembly installed onto an imaging device according to at least one embodiment of the present disclosure;

FIG. 6B is a side view of an adaptor and a corrector assembly installed onto an imaging device according to at least one embodiment of the present disclosure;

FIG. 7A is a perspective view of an adaptor and a corrector assembly according to at least one embodiment of the present disclosure;

FIG. 7B is a side view of an adaptor and a corrector assembly according to at least one embodiment of the present disclosure;

FIG. 8 is a top view of an adaptor and a corrector assembly according to at least one embodiment of the present disclosure;

FIG. 9A is a perspective view of a bracket according to at least one embodiment of the present disclosure;

FIG. 9B is a side view of a bracket according to at least one embodiment of the present disclosure;

FIG. 9C is a rear view of a bracket according to at least one embodiment of the present disclosure;

FIG. 10A is a perspective view of a side bracket according to at least one embodiment of the present disclosure;

FIG. 10B is a perspective view of a side bracket according to at least one embodiment of the present disclosure;

FIG. 11A is a perspective view of an adaptor and a corrector assembly according to at least one embodiment of the present disclosure;

FIG. 11B is a side view of an adaptor and a corrector assembly according to at least one embodiment of the present disclosure;

FIG. 12 is a top view of an adaptor and a corrector assembly according to at least one embodiment of the present disclosure;

FIG. 13A is a perspective view of a clamping assembly according to at least one embodiment of the present disclosure;

FIG. 13B is a close-up perspective view of a clamping assembly according to at least one embodiment of the present disclosure;

FIG. 13C is a close-up perspective view of a clamping assembly according to at least one embodiment of the present disclosure;

FIG. 14A is a perspective view of a clamping assembly according to at least one embodiment of the present disclosure;

FIG. 14B is a perspective view of a clamping assembly according to at least one embodiment of the present disclosure;

FIG. 15 is a perspective, partially exploded view of some components of a clamping assembly according to at least one embodiment of the present disclosure;

FIG. 16A is a perspective view of a clamping arm according to at least one embodiment of the present disclosure;

FIG. 16B is a cross-section side view of a clamping arm according to at least one embodiment of the present disclosure;

FIG. 17A is a cross-section side view of a clamping arm in a first position according to at least one embodiment of the present disclosure;

FIG. 17B is a cross-section side view of a clamping arm in a second position according to at least one embodiment of the present disclosure;

FIG. 18A is a side view of an adaptor and a corrector assembly according to at least one embodiment of the present disclosure;

FIG. 18B is a top view of an adaptor and a corrector assembly according to at least one embodiment of the present disclosure;

FIG. 19A is a perspective view of a belt assembly according to at least one embodiment of the present disclosure;

FIG. 19B is a perspective view of a belt assembly according to at least one embodiment of the present disclosure;

FIG. 19C is a perspective view of a belt assembly according to at least one embodiment of the present disclosure;

FIG. 20 is an exploded perspective view of a ratchet according to at least one embodiment of the present disclosure;

FIG. 21A is a perspective view of a ratchet according to at least one embodiment of the present disclosure;

FIG. 21B is a cross-section front view of a ratchet according to at least one embodiment of the present disclosure;

FIG. 22 is a side view of an adaptor and a corrector assembly installed on an imaging device according to at least one embodiment of the present disclosure;

FIG. 23 is a block diagram of a system according to at least one embodiment of the present disclosure; and

FIG. 24 is a flow chart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors: AMD Athlon processors; AMD Phenom processors; Apple A10 or 10X Fusion processors: Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

With the development of radiography and related imaging devices (e.g., C-arm, DSA, CT, MR, etc.) an orthopedic surgeon is able to view orthopedic characteristics of a patient and locate implants (e.g., a pedicle screw, interbody position) that may have been inserted into a patient. A mobile C-arm is often used in orthopedic surgery and is prevalent in many hospitals. The mobile C-arm enables a surgeon to view real-time imaging of a patient, and may assist a surgeon in conducting accurate surgical procedures such as, for example, a reduction, sub-millimeter screw-driving, and implantation of an implant.

Moreover, in robotic assisted surgery, the mobile C-arm has additional applications. For instance, a surgeon may use a CT exam to form a pre-operative surgical plan including determining an implant selection, implant trajectory, operation procedure, and an after-surgical estimation. During the surgery, the real-time patient's position may be acquired by the mobile C-arm and subsequently one or more images may be obtained which are correlated with a reconstructed CT exam projection. This process is called registration, which may have two methods. A first method (which may be referred to as “Scan & Plan”) uses intra-operative O-arm 3D images. A second method (which may be referred to as “CT to Fluro”) uses pre-operative CT 3D images and intra-operative C-arm 2D images. However, the C-arm images may be distorted, thereby increasing difficulties in image segmentation and registration. As such, a conventional solution to such distortions is to install a C-arm image corrector to an intensifier to calibrate the distorted image.

Typically, the C-arm may have two types of detector structures. A first type is round and a second type is a flat panel. For the round types, a diameter of the intensifier may be about 9″ or about 12″. For the flat panel types, a width and a height of the flat panel may be about 20 cm×20 cm or about 30 cm×30 cm. Typically, all flat panel type C-arm models in the markets have handle rails on three sides. A problem with conventional mobile C-arms is that different manufactures manufacture C-arms of various types, sizes, and shapes. Thus, an adaptor designed to ensure ease of installation and fitting all types, sizes, and shapes of C-arms is beneficial.

At least one embodiment of the present disclosure provides for a crane adaptor that can enable an operator to assemble or install a corrector to different types of mobile C-arms more easily. The adaptor may comprise a main clamping rod and at least one side clamping rod. During installation, a user may hold the main clamping rod up to a specific height over the C-arm and use another hand to tighten a strap between the main clamping rod and the at least one side clamping rod to securely attach the adaptor to the C-arm.

At least one embodiment of the present disclosure provides for an adaptor comprising four brackets, each bracket having a lateral structure operable to adjust in height relative to the bracket. The lateral structure may be tightened against the C-arm to secure the adaptor to the C-arm.

At least one embodiment of the present disclosure provides for an adaptor comprising three clamps adjustable laterally, rotationally, and longitudinally. The adaptor may comprise an adjuster operable to enable adjustment of a height of a clamp. The adjuster may comprise a threaded bolt received in a half threaded slot hole that can achieve an adjustment function in a lateral direction.

At least one embodiment of the present disclosure provides for a belt adaptor comprising three belts with a rachet tight function to attach the adaptor to the C-arm handle rails. Since flat panel C-arm models typically have three-sided handle rails, the adaptor may comprise three belts with a rachet to attach and tighten the adaptor to different flat panel C-arm models handle rails in three directions. The belt adaptor may comprise a ratchet designed to limit a force applied to the C-arm detector when the ratchet is used to tighten the belt(s) to the C-arm. The ratchet may comprise one or more springs operable to prevent an applied force greater than a threshold force from being applied to the C-arm detector.

Embodiments of the present disclosure provide technical solutions to one or more of the problems of (1) providing an adaptor capable of installation onto multiple shapes, sizes, and types of imaging devices: (2) adjusting a plurality of holders of an adaptor in one or more directions to enable installation of the adaptor onto multiple shapes, sizes, and types of imaging devices: (3) installing an adaptor onto an imaging device; and (4) improving an efficiency and speed of installing an adaptor onto an imaging device.

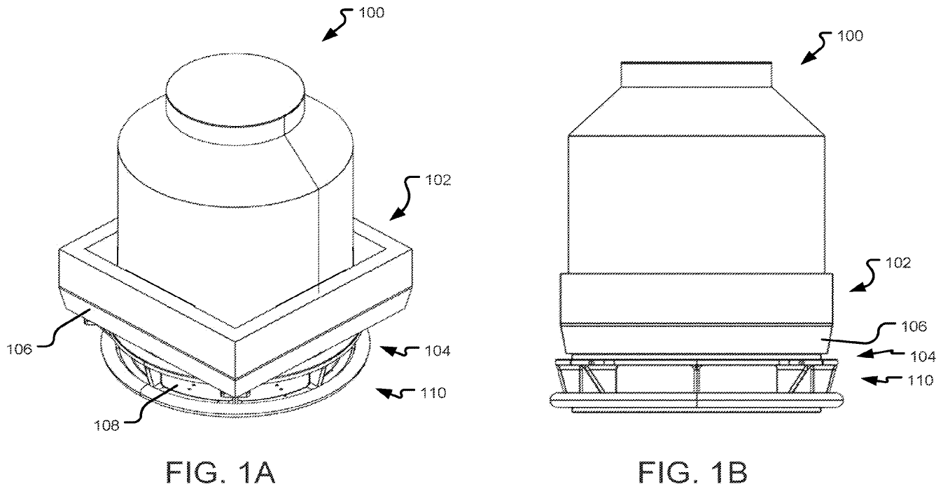

Turning first to FIGS. 1A and 1B, a perspective view and a side view, respectively, of a detector 102 of an imaging device 100 and a corrector assembly 104 are shown. The imaging device 100 may be, for example, an X-ray device. In the illustrated example, the X-ray device includes the detector 102 supported at one end of a C-arm device, and a source (not shown) supported at an opposite end of the C-arm device. It will be appreciated that in other embodiments, the detector 102 may be supported by, for example, a first robotic arm, and the source may be supported by, for example, a second robotic arm. In some applications, an image obtained from the imaging device 100 may be distorted and cause issues with, for example, a registration process. As such, at least one corrector 108 of the corrector assembly 104 is installed onto the detector 102, and more specifically, to an intensifier 106 of the detector 102 to calibrate an image obtained from the imaging device 100. The corrector 108 may be in the form of a plate. As shown, the corrector 108 is shown mated to a front of the intensifier 106 by a mounting ring 110 of the corrector assembly 104. The intensifier 106 may be, in some embodiments, circular, whereas in other embodiments such as the illustrated embodiment, the intensifier 106 may be a flat-panel.

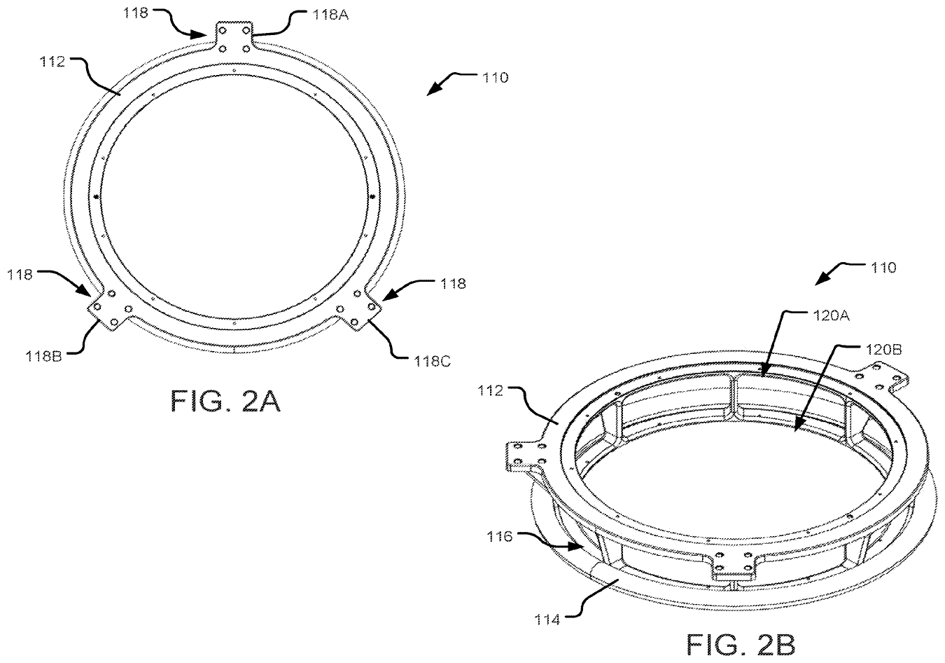

Turning to FIGS. 2A and 2B, a top view and a perspective view, respectively, of the mounting ring 110 are shown. The mounting ring 110 comprises an upper ring 112 and a lower ring 114 connected by a plurality of cross members 116. A distance between the upper ring 112 and the lower ring 114 may be based on a spacing determined between the corrector 108 and the intensifier 106 to achieve calibration. The upper ring 112 and the lower ring 114 each comprise an opening 120A, 120B. The openings 120A, 120B are each configured to receive the corrector 108 (whether one opening receives a corrector, or both openings each receive a corrector). In the illustrated embodiment, the openings 120A, 120B are circular, though in other embodiments, the openings 120A, 120B may be any shape such as, for example, rectangular, square, or oval. It will be appreciated that in some embodiments the openings 120A, 120B may each be a different shape from each other.

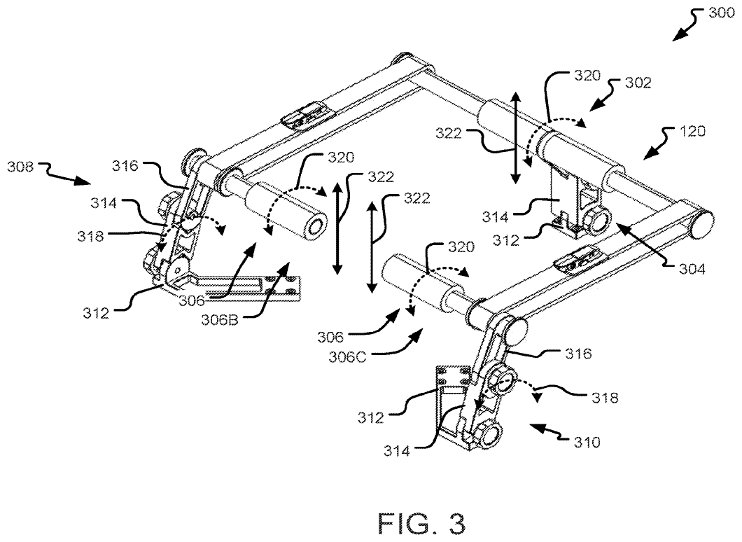

The upper ring 112 includes a plurality of projections 118, and as illustrated, the plurality of projections 118 comprises three projections 118A (e.g., a first projection), 118B (e.g., a second projection), 118C (e.g., a third projection). It will be appreciated that in other embodiments, the plurality of projections 118 may comprise greater or less than three projections. In the illustrated embodiment, projections 118B and 118C are equally spaced from projection 118A. In other instances, the projections 118A, 118B, and 118C may be spaced any distance from each other. The plurality of projections 118 are configured to receive a plurality of holders 120 (shown in FIGS. 3, 7A, 1100A, and 1800A) of an adaptor (such as an adaptor 300, 700, 1100, 1800 described below) to secure the adaptor to the mounting ring 110, which will be described in detail below. The plurality of holders 120 are configured to attach to the intensifier 106 to secure the corrector assembly 104 to the intensifier 106. The plurality of holders 120 are configured to adjust in one or more directions.

With reference to FIGS. 3-6B, an adaptor 300 according to at least one embodiment of the present disclosure is shown.

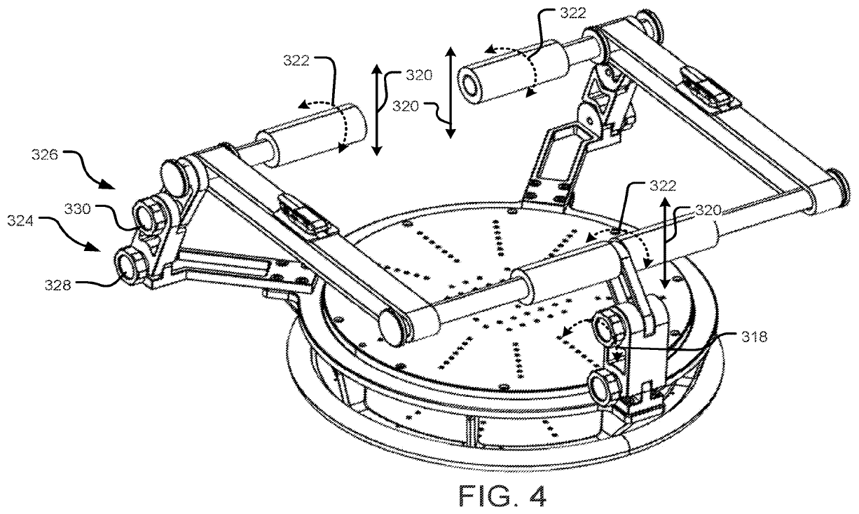

FIG. 3 shows a front perspective view of the adaptor 300 and FIG. 4 shows a rear perspective view of the adaptor 300 and the corrector assembly 104. The adaptor 300 comprises the plurality of holders 120. The plurality of holders 120 comprise a clamping bar 302 supported by a first arm 304 and at least one side bar 306. In the illustrated embodiment the at least one side bar 306 comprises a first side bar 306A supported by a second arm 308 and a second side bar 306B supported by a third arm 310. It will be appreciated that in other embodiments, the at least one side bar 306 may comprise one, two, or more than two side bars.

Each of the first arm 304, the second arm 308, and the third arm 310 comprise a base 312, a first link 314 pivotably connected to the base 312, and a second link 316 pivotably connected to the first link 314 at one end and to the clamping bar 302 at another end. The base 312, the first side bar 306A, the second side bar 306B, the clamping bar 302, the first link 314, and the second link 316 may be each formed from a lightweight solid material such as for example, an aluminum alloy, or polyether ether ketone (PEEK). The base 312 is configured to couple the adaptor 300 to the mounting ring 110. The base 312 of each of the first arm 304, the second arm 308, and the third arm 310 may be secured to a corresponding protrusion 118A, 118B, 118C of the mounting ring 110 by using, for example, bolts and nuts, screws, rivets, adhesion, or the like. In some embodiments, the base 312 may be formed as a part of the mounting ring 110.

The first link 314 and the second link 316 are pivotable. The first link 314 is pivotable with respect to the base 312 and causes the second link 316 to move along a first arc 318 when the first link 314 is pivoted. The second link 316 is pivotable with respect to the first link 314 and causes the corresponding clamping bar 302, first side bar 306A, or second side bar 306B to move along a second arc 320 when the second link 316 is pivoted. Due to the pivoting motion of both the first link 314 and the second link 316, the corresponding clamping bar 302, first side bar 306A, or second side bar 306B may also move translationally in the direction of the arrow 322. In other words, the clamping bar 302, first side bar 306A, or second side bar 306B may move rotationally and/or translationally. Such motion allows for the clamping bar 302, first side bar 306A, or second side bar 306B to be moved and adjusted to fit over a variety of shapes and sizes of intensifiers 106.

In some embodiments, a first stopper 324 and a second stopper 326 may be disposed at a pivot point of the first link 314 and the second link 316, respectively. The first stopper 324 and the second stopper 326 may each be configured to lock the first link 314 and the second link 316, respectively, when actuated. In some embodiments, the first stopper 324 and the second stopper 326 comprise a knob 328, 330 and a threaded protrusion (not visible). When the knob 328, 330 is rotated in a first direction, the threaded protrusion may thread and move into a receiver (not visible), which pushes the corresponding first link 314 or second link 316 into the receiver. This rotation restricts movement of the first link 314 or the second link 316 by compressing the first link 314 or the second link 316 between the knob 328, 330 and the receiver. When the knob 328, 330 is rotated in a second direction opposite the first direction, the threaded protrusion may be unthreaded from the receiver and may move away from the receiver, thereby loosening the corresponding first link 314 or second link 316. It will be appreciated that in some embodiments, the adaptor 300 may not include the first stopper 324 and/or the second stopper 326. In such embodiments, the first link 314 and the second link 316 may be free to pivot.

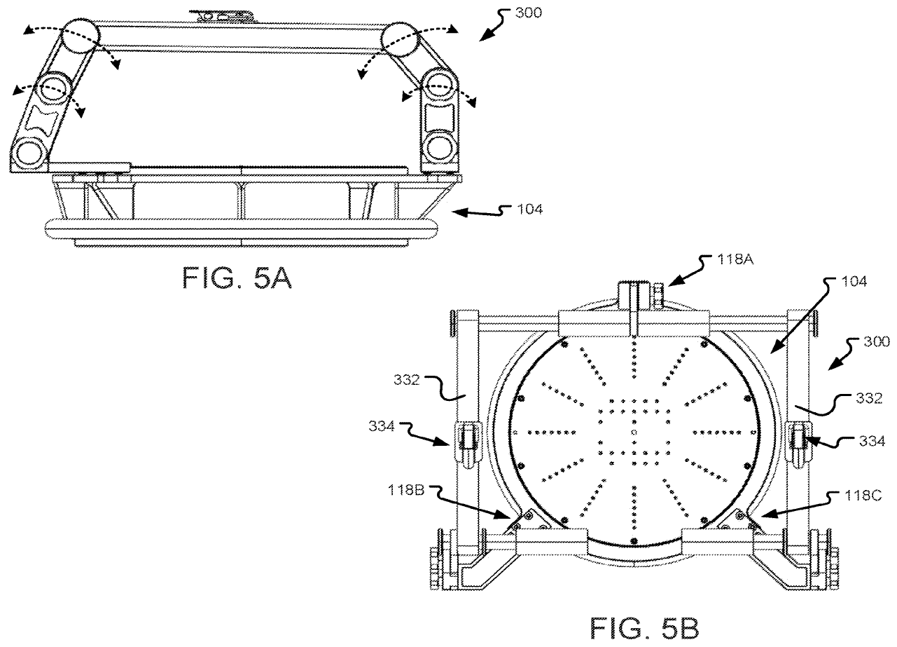

Turning to FIGS. 5A and 5B, a side view and a top view, respectively, of the adaptor 300 and the corrector assembly 104 are shown. As previously described and shown, the first link 314 and the second link 316 are pivotable, as also shown in FIG. 5A. As illustrated in FIG. 5B, the first side bar 306A and the second side bar 306B may each have a length less than the clamping bar 302. It will be appreciated that in some embodiments, each of the first side bar 306A and/or the second side bar 306B may have a length equal to or greater than the clamping bar 302. Also illustrated, the first arm 304 is secured to protrusion 118A, the second arm 308 is secured to protrusion 118B, and the third arm 310 is secured to protrusion 118C. It will be appreciated that the first arm 304, the second arm 308, and the third arm 310 may be fixed to any protrusion 118A, 118B, 118C. Further, as illustrated in FIGS. 5A and 5B, the first side bar 306A and the second side bar 306B may be connected to the clamping bar 302 by a corresponding strap 332. Each strap 332 may be adjustable and tightened by an adjuster 334. In some embodiments, each strap 332 is formed from a material such as, for example, nylon, webbing, or the like. In other instances, each strap 332 may formed from a resilient material such as, for example, a rubber band, that is biased to an initial position of the band. The strap 332 may be used to tighten and secure the clamping bar 302, the first side bar 306A, and the second side bar 306B to the intensifier 106.

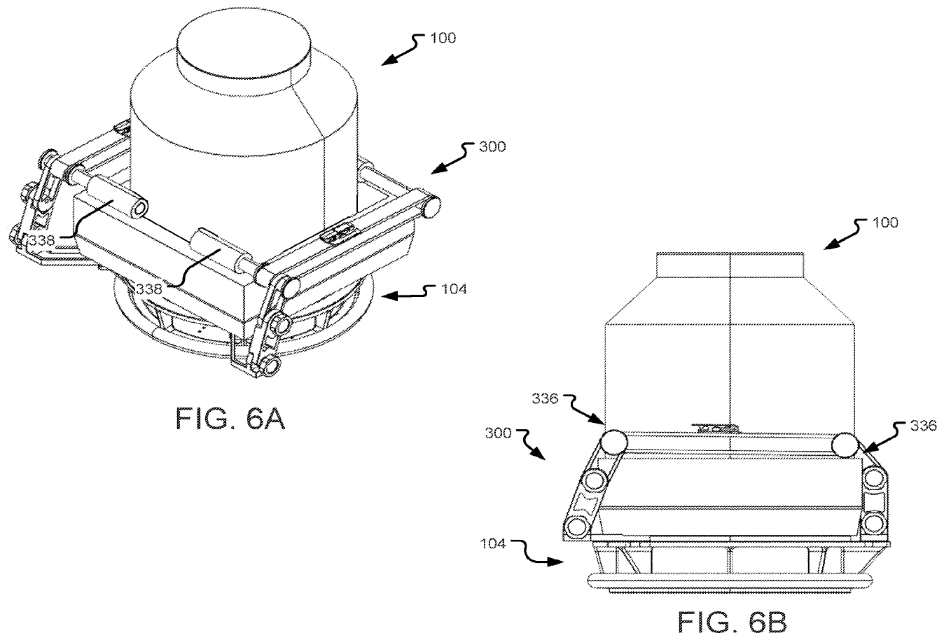

FIGS. 6A and 6B illustrate a perspective view and a side view, respectively, of the adaptor 300 fixed to the intensifier 106. As shown, the adaptor 300 is secured or fixed to the intensifier 106 at a plurality of contact points 336. In some embodiments, a rubber cover or pad 338 may be fixed to the clamping bar 302, the first side bar 306A, and the second side bar 306B to increase a contact area and/or grip between the adaptor 300 and the intensifier 106 and/or to prevent damage to the intensifier 106. The rubber cover or pad 338 may be formed from, for example, a compound rubber such as polybutadiene rubber.

During installation, the clamping bar 302 is held over the intensifier 106 while one of the straps 332 is tightened (whether by the adjuster 334 or simply moving the bar(s) 302, 306A, 306B in place in the case of resilient strap(s)). Tightening of the strap 332 may move the corresponding first side bar 306A or second side bar 306B into one of the plurality of contact points 336 until the first side bar 306A or the second side bar 306B (and the clamping bar 302) are secured to the intensifier 106. The process may be repeated (wherein another of the straps 332 is tightened) to secure the other side bar of the first side bar 306A or the second side bar 306B to the intensifier 106.

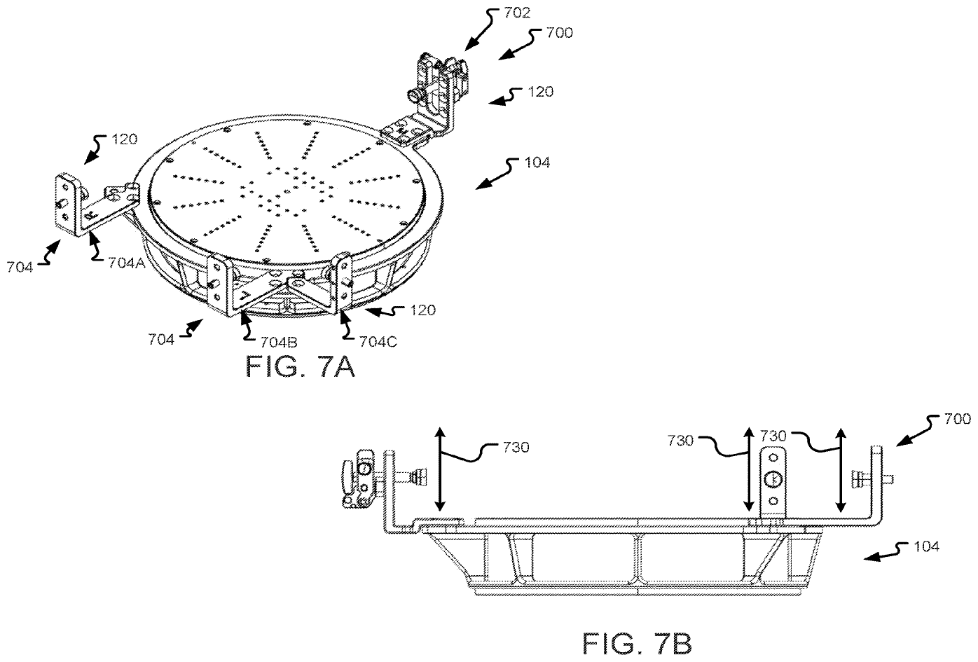

With reference to FIGS. 7A-10B, an adaptor 700 according to at least one embodiment of the present disclosure is provided.

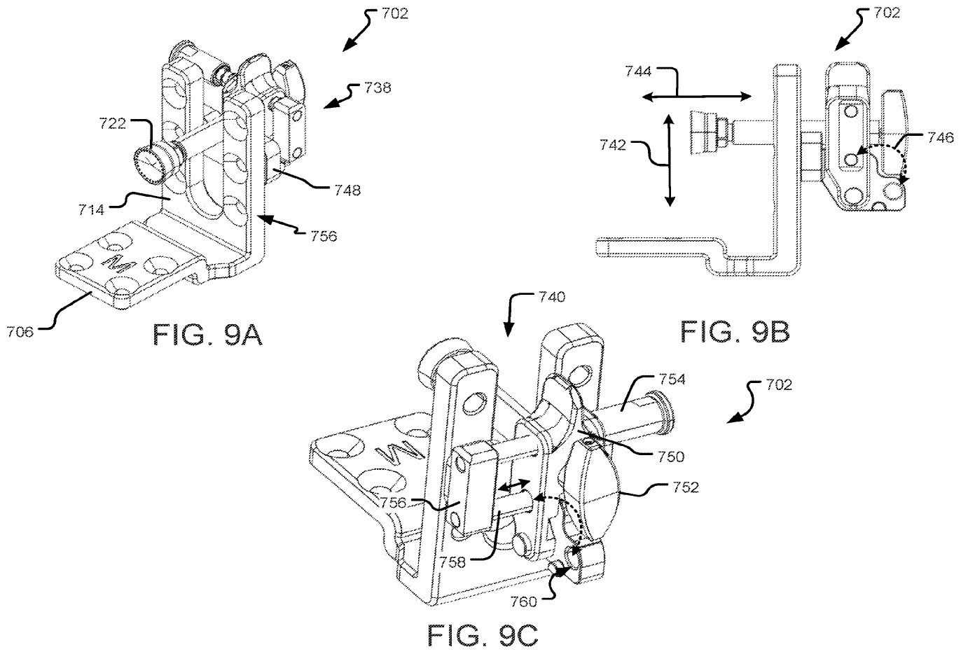

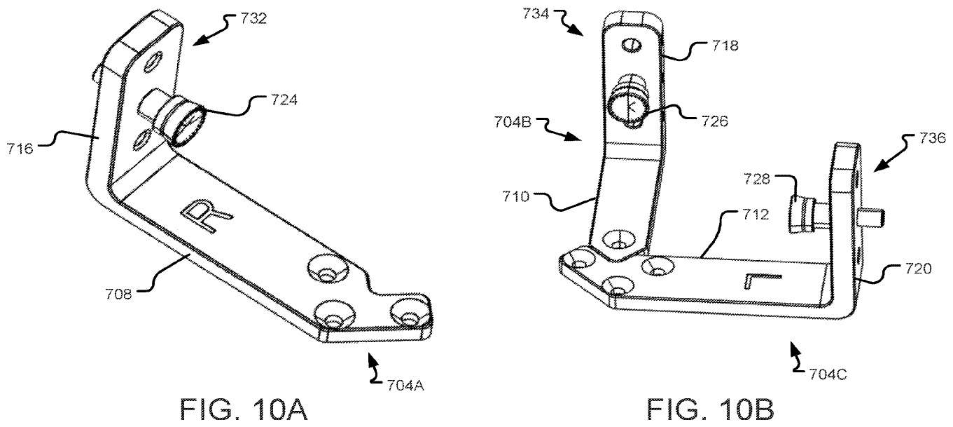

FIGS. 7A and 7B show a perspective view and a side view, respectively, of the adaptor 700 fixed to the corrector assembly 104. The adaptor 700 comprises the plurality of holders 120. The plurality of holders 120 comprises a bracket 702 and at least one side bracket 704. In the illustrated embodiment, each of the bracket 702 and the at least one side bracket 704 is fixed to the mounting ring 110. It will be appreciated that in other embodiments the bracket 702 and/or one or more of the at least one side bracket 704 may be rotatable with respect to the mounting ring 110. In the illustrated embodiment, the at least one side bracket 704 comprises a first side bracket 704A, a second side bracket 704B, and a third side bracket 704C. In other embodiments, the at least one side bracket 704 may comprise one, two, or more than two side brackets. Each of the bracket 702 and the at least one side bracket 704 comprises a base 706, 708, 710, 712 (referenced in FIGS. 9A-10B) securable to the mounting ring 110 and a wall 714, 716, 718, 720 perpendicular to the base 706, 708, 710, 712. The base 706, 708, 710, 712 may be secured to a corresponding protrusion 118A, 118B 118C by using, for example, bolts and nuts, screws, rivets, adhesion, or the like.

The plurality of holders 120 also comprises a corresponding spacer 722, 724, 726, 728 for each of the bracket 702, the first bracket 702, the second bracket 702, and the third bracket 702. The spacer 722, 724, 726, 728 is adjustable in a lateral direction, shown by the arrow 730, to adjust a height of the spacer 722, 724, 726, 728 relative to the mounting ring 110. The spacer 724, 726, 728 is adjusted by securing the spacer 724, 726, 728 in one of a plurality of apertures 732, 734, 736 disposed on the wall 716, 718, 720. The spacer 722 is adjustable in the lateral direction on the bracket 702 by an adjuster 738.

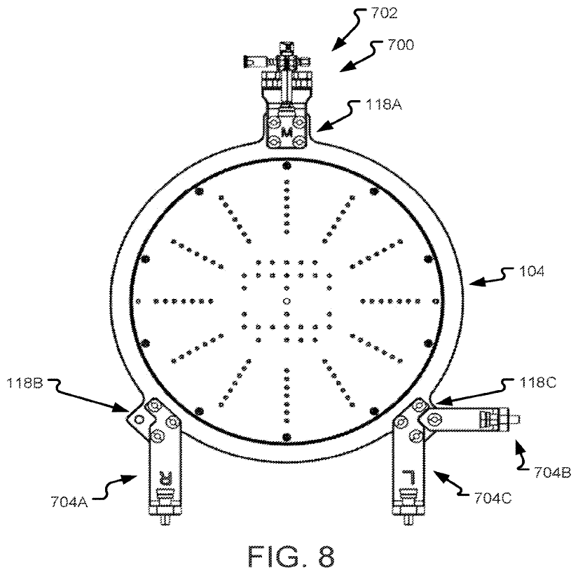

Turning to FIG. 8, a top view of the adaptor 700 and the corrector assembly 104 are shown. As shown, the bracket 702 is fixed to the protrusion 118A, the first side bracket 704A is fixed to the protrusion 118B, and the second side bracket 704B and the third side bracket 704C are fixed to the protrusion 118C. The second side bracket 704B and the third side bracket 704C are positioned such that their respective bases 710, 712 are perpendicular to each other. It will be appreciated that the bracket 702, the first side bracket 704A, the second side bracket 704B, and the third side bracket 704C may be fixed to any protrusion 118A, 118B, 118C and in any orientation. The arrangement of the bracket 702, the first side bracket 704A, the second side bracket 704B, and the third side bracket 704C combined with the spacers 722, 724, 726, 728 that are adjustable in the lateral direction provide for an adaptor 700 that can be fixed to intensifiers of multiple sizes and shapes.

Turning to FIGS. 9A-9C, a first perspective view of the bracket 702, a side view of the bracket 702, and a second perspective view of the bracket 702 are respectively shown. The wall 714 includes a slot 740 through which the spacer 722 extends through. The adjustor 738 is configured to adjust the spacer 722 laterally as depicted by arrow 742, longitudinally as depicted by arrow 744, or rotationally as depicted by arrow 746. In the illustrated embodiment, the adjustor 738 comprises an adjustor base 748, a lever 750, a knob 752, and a trigger 754. The spacer 722 may be adjusted laterally by moving the adjustor base 748 laterally and fixing the adjustor base 748 to one of a plurality of apertures 756 disposed on the wall 714 (whether by screws, bolts, rivets, adhesion, or otherwise). The spacer 722 may be adjusted longitudinally by the knob 752. In some embodiments, the spacer 722 may include a threaded outer surface on a shaft that is received by a threaded aperture of the lever 750. In such embodiments, when the knob 752 is rotated in a first direction, the spacer 722 moves longitudinally in a first direction and when the knob 752 is rotated in a second direction, the spacer 722 moves longitudinally in a second direction.

The spacer 722 may be rotationally adjusted by the trigger 754. In some embodiments, the trigger 754 is biased by, for example, a spring (not visible), away from a trigger base 756. The trigger base 756 includes a protrusion 758 receivable in a first aperture (not visible) or a second aperture 760. When the trigger 754 is pushed against the bias, the trigger base 756 and the protrusion 758 are pushed away from the lever 750 and the protrusion 758 is pushed out of the first aperture. When the trigger 754 is rotated, the trigger base 756 and the protrusion 758 are rotated or pivoted until the protrusion 758 is aligned with the second aperture 760. When the trigger 754 is released, the bias pushed the trigger 754 away from the lever 750, thereby pulling the trigger base 756 towards the lever 750 and the protrusion 758 into the second aperture 760.

Turning to FIGS. 10A-10B, a perspective view of the first side bracket 704A and a perspective view of the second side bracket 704B and the third side bracket 704C are shown, respectively. As previously described, the spacers 724, 726, 728 are laterally adjustable. In some embodiments, the spacers 724, 726, 728 include a threaded shaft and the plurality of apertures 732, 734, 736 may be threaded to receive the threaded shaft. In such embodiments, each spacer 724, 726, 728 may be simply unthreaded from an aperture and threaded into another aperture to move the spacer 724, 726, 728 laterally along the wall 716, 718, 720. It will be appreciated that in other embodiments, each spacer 724, 726, 728 may be laterally adjusted in any way, for example, by sliding the spacer 724, 726, 728 along the wall 716, 718, 720, or otherwise. The spacers 724, 726, 728 may also be laterally, longitudinally, or rotationally adjusted by, for example, a corresponding adjustor such as the adjustor 738 described above.

During installation the spacers 722, 724, 726, 728 may be laterally adjusted along the wall 714, 716, 718, 720 based on a height of the intensifier 106. The spacer 722 may be initially rotated or pivoted away from the mounting ring 110 so that the intensifier 106 can be positioned between the spacers 722, 724, 726, 728. When the intensifier 106 is in place, the spacer 722 may be pivoted back towards the mounting ring 110 so as to contact and press against the intensifier 106 to lock the adaptor 700 to the intensifier 106. It will be appreciated that in other embodiments, the adaptor 700 may be secured to the intensifier 106 with greater or less than four spacers. Though not shown, the adaptor 700 is secured or fixed to the intensifier 106 at a plurality of contact points. The spacers 722, 724, 726, 728 may also comprise a rubber cover or pad to increase a contact area and/or grip between the adaptor 700 and the intensifier 106 and/or to prevent damage to the intensifier 106. The rubber cover or pad may be formed from, for example, a compound rubber such as polybutadiene rubber.

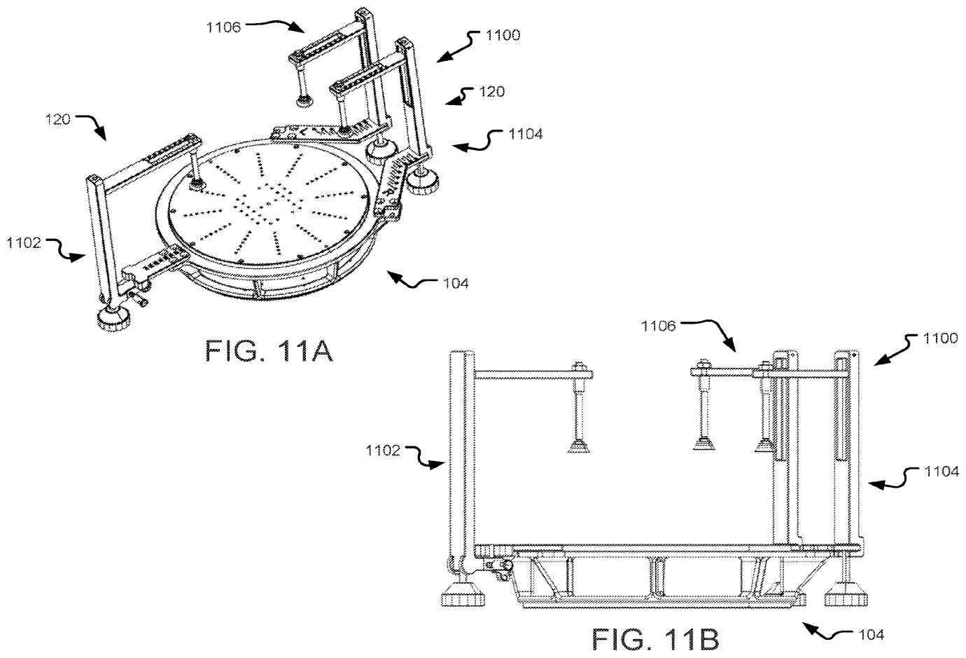

With reference to FIGS. 11A to 17B, an adaptor 1100 according to at least one embodiment of the present disclosure is provided.

FIGS. 11A and 11B show a perspective view and a side view, respectively, of the adaptor 1100 fixed to the corrector assembly 104. The adaptor 1100 comprises the plurality of holders 120. In the illustrated embodiment, the plurality of holders 120 comprises a first clamp assembly 1102, a second clamp assembly 1104, and a third clamp assembly 1106. In other embodiments, the plurality of holders 120 may comprise less or greater than three clamp assemblies. Each clamp assembly 1102, 1104, 1106 comprises a clamp base 1108, 1110, 1112, a clamp bracket 1114, 1116, 1118 extending from the clamp base 1108, 1110, 1112, a clamp arm 1120, 1122, 1124 extending from the clamp bracket 1114, 1116, 1118, and a spacer 1126, 1128, 1130 supported by the clamp arm 1120, 1122, 1124. Each clamp base 1108, 1110, 1112 is attached or fixed to each protrusion 118A, 118B, 118C of the mounting ring 110, thereby connected the adaptor 1100 to the corrector assembly 104.

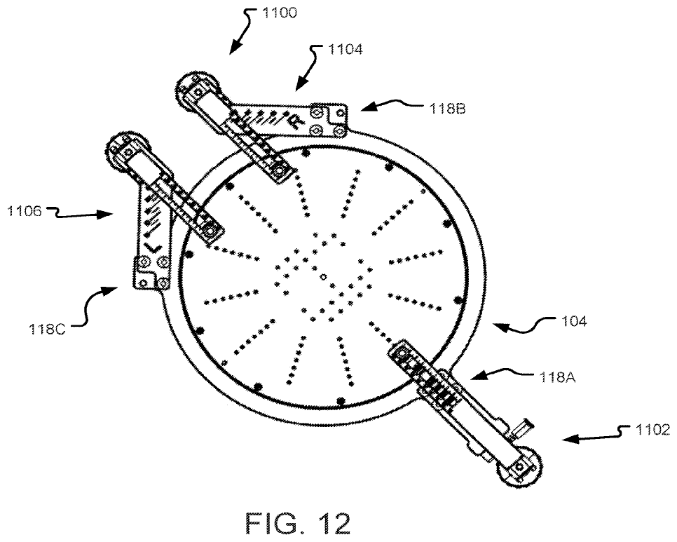

Turning to FIG. 12, a top view of the adaptor 1100 fixed to the corrector assembly 104 is shown. As shown, the first clamp assembly 1102 is fixed to the first protrusion 118A, the second clamp assembly 1104 is fixed to the second protrusion 118B, and the third clamp assembly 1106 is fixed to the third protrusion 118C. It will be appreciated that in other embodiments, the first clamp assembly 1102, the second clamp assembly 1104, and/or the third clamp assembly 1106 may be fixed to any protrusion 118A, 118B, 118C.

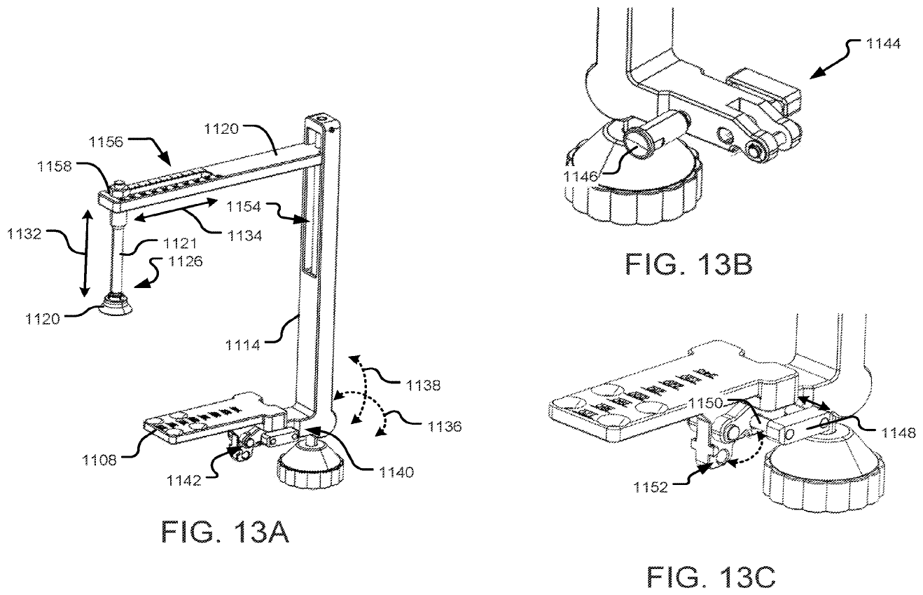

Turning to FIGS. 13A-13C, a perspective view of the first clamp assembly 1102, a close-up perspective view of a portion of the first clamp assembly 1102 (the base 1108 is not shown for clarity), and another close-up perspective view of a portion of the first clamp assembly 1102 are shown, respectively. As shown, the spacer 1126 is adjustable in a lateral direction as depicted by arrow 1132, a longitudinal direction as depicted by arrow 1134, rotationally as depicted by arrow 1136, and pivotable as depicted by arrow 1138. As previously described, the base 1108 is secured to the protrusion 118A by, for example, screws, bolts, riveting, or otherwise. The base 1108 may be rotatable in the direction of the arrow 1136 based on a position of the base 1108 when secured to the protrusion 118A. In other words, the base 1108 (and thus, the first clamp assembly 1102) may be rotated to a desirable position on the protrusion 118A before being secured to the protrusion 118A. As shown, the clamp bracket 1114 may extend perpendicularly from the base 1108. The clamp bracket 1114 may have an extension 1140 secured to the base 1108 at a pivotable joint 1142. The clamp bracket 1114 may be pivotable in the direction of the arrow 1138 using an adjuster 1144.

The adjuster 1144 may be the same as the adjuster 738 described above with respect to FIGS. 9A-9C. More specifically, the adjuster 1144 may include a trigger 1146, visible in FIG. 13B. In some embodiments, the trigger 1146 is biased by, for example, a spring, away from a trigger base 1148. The trigger base 1148 includes a protrusion 1150 receivable in a first aperture (not visible) or a second aperture 1152 of the base 1108. When the trigger 1146 is pushed against the bias, the trigger base 1148 and the protrusion 1150 are pushed away from the base 1108 and the protrusion 1150 is pushed out of the first aperture. When the trigger 1146 is rotated, the trigger base 1148 and the protrusion 1150 are rotated or pivoted until the protrusion 1150 is aligned with the second aperture 1152. When the trigger 1146 is released, the bias pushes the trigger 1146 away from the base 1108, thereby pulling the trigger base 1148 towards the base 1108 and the protrusion 1150 into the second aperture 1152.

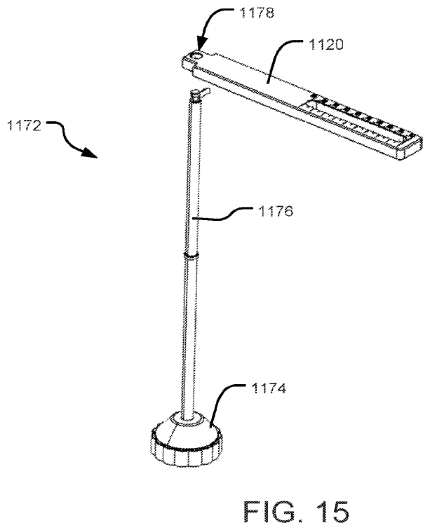

The first clamp assembly 1102 also includes the clamp arm 1120 which extends perpendicularly from the clamp bracket 1114. The clamp arm 1120 is adjustable in the direction of the arrow 1132 along a first slot 1154 of the clamp bracket 1114, which adjusts a height of the spacer 1126. The adjustability of the clamp arm 1120 in the lateral direction will be discussed with respect to FIGS. 15-17B. The clamp arm 1120 also includes a second slot 1156 through which the spacer 1126 extends through. The spacer 1126 is adjustable in the longitudinal direction along the second slot 1156. The spacer 1126, in some embodiments, may include a threaded portion. In such embodiments, to adjust the spacer 1126, a nut 1158 disposed at a top of the spacer 1126 may be loosened or removed from the threaded portion, the spacer 1126 may be slid along the second slot 1156 to the desired position, and the nut 1158 may then be threaded onto the threaded portion to secure the spacer 1126 in place. The spacer 1126 may also comprise a shaft 1121 and a contact end 1158 connected to an end of the shaft 1121 in a ball and joint configuration so that the contact end 1158 is swivelable with respect to the shaft 1121. The contact end 1158 may include a rubber cover or pad to increase a contact area and/or grip between the adaptor 1100 and the intensifier 106 and/or to prevent damage to the intensifier 106. The rubber cover or pad may be formed from, for example, a compound rubber such as polybutadiene rubber.

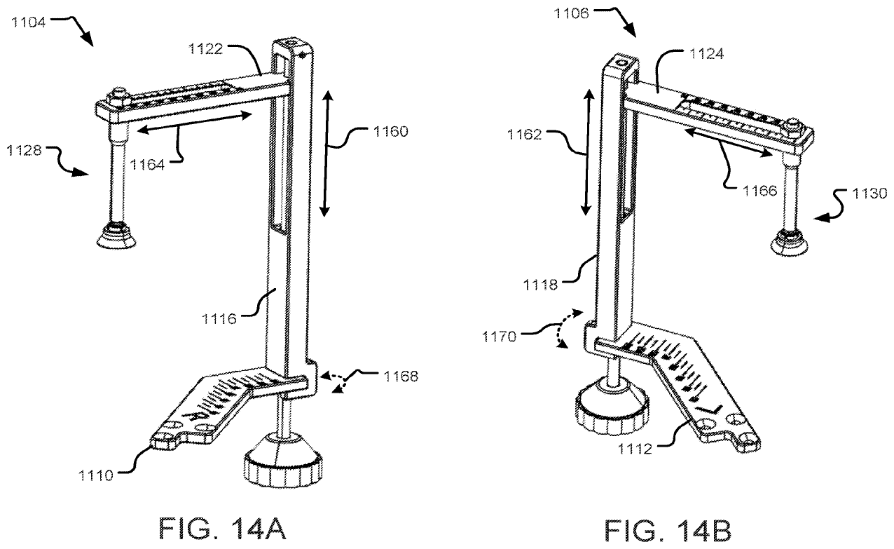

Turning to FIGS. 14A-14B, a perspective view of the second clamp assembly 1104 and a perspective view of the third clamp assembly 1106 are respectively shown. The second clamp assembly 1104 and the third clamp assembly 1106 are adjustable in the same or similar ways as described above with respect to the first clamp assembly 1102, except that the second clamp assembly 1104 and the third clamp assembly 1106 do not include an adjustor to pivotably adjust the spacers 1128, 1130, respectively. It will be appreciated that in some embodiments, the second clamp assembly 1104 and/or the third clamp assembly 1106 may include an adjustor to pivotably adjust the spacers 1128, 1130. As shown, the spacers 1128, 1130 are adjustable in a lateral direction as depicted by arrow 1160, 1162, a longitudinal direction as depicted by arrow 1164, 1166, and rotationally as depicted by arrow 1168, 1170. The spacers 1128, 1130 are adjustable using the same mechanisms described above with respect to the first clamp assembly 1102.

As illustrated, the base 1108, 1110, 1112 and the clamp arm 1114, 1116, 1118 each include a scale line for visually adjusting the respective clamp assemblies 1102, 1104, 1106. It will be appreciated that any component described herein (whether a clamp arm, a bracket, a clamp rod, etc.) may include such indicia for measuring, adjusting, or moving a spacer.

Turning to FIG. 15, a perspective view of the clamp arm 1120 and a lateral adjustor 1172 according to at least one embodiment of the present disclosure is provided. Though the lateral adjustor 1172 is described with respect to the first clamp assembly 1102, it will be appreciated that the lateral adjustor 1172 may be used with the second clamp assembly 1104 and/or the third clamp assembly 1106. In some embodiments, the clamp arm 1120 is adjustable by the lateral adjustor 1172, which comprises a knob 1174 and a threaded shaft 1176 extending from the knob 1174. The thread is disposed on an outer surface of the shaft 1176. The threaded shaft 1176 extends through the clamp bracket 1114, though the threaded shaft 1176 may simply extend parallel to the clamp bracket 1114. The clamp arm 1120 may have a corresponding threaded aperture 1178 that receives the threaded shaft 1176. When the knob 1174 is rotated in a first direction, the clamp arm 1120 may move in a first lateral direction and when the knob 1174 is rotated in a second direction, the clamp arm 1120 may move in a second lateral direction opposite the first lateral direction.

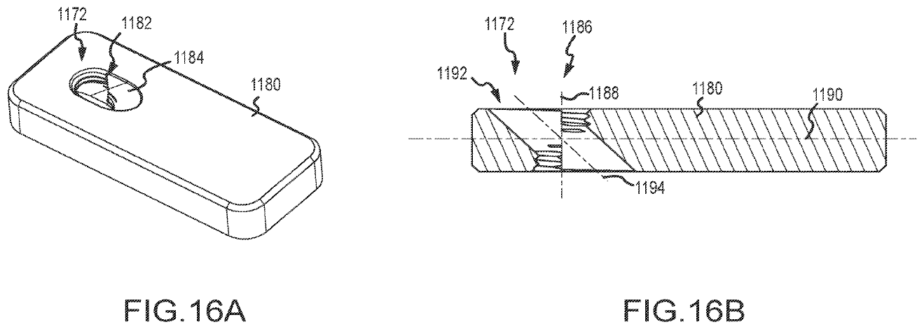

Turning to FIGS. 16A and 16B, a first perspective view of another embodiment of a clamp arm 1180 and a cross-section side view of the clamp arm 1180 are respectively provided. It will be appreciated that the clamp arm 1180 may have a spacer and a slot similar to or the same as the spacer 1126, 1128, 1130 and the second slot 1156, 1158, 1160 of the clamp arm 1120, 1122, 1124. As shown, the clamp arm 1180 includes a lateral adjustor 1172 comprising an aperture 1182 having an inner surface 1184. As illustrated in FIG. 16B, a profile of the inner surface 1184 includes a threaded bore 1186 having an axis 1188 perpendicular to a center line 1190. The inner surface 1184 also includes a non-threaded bore 1192 having an axis 1194 oblique to the center line 1190.

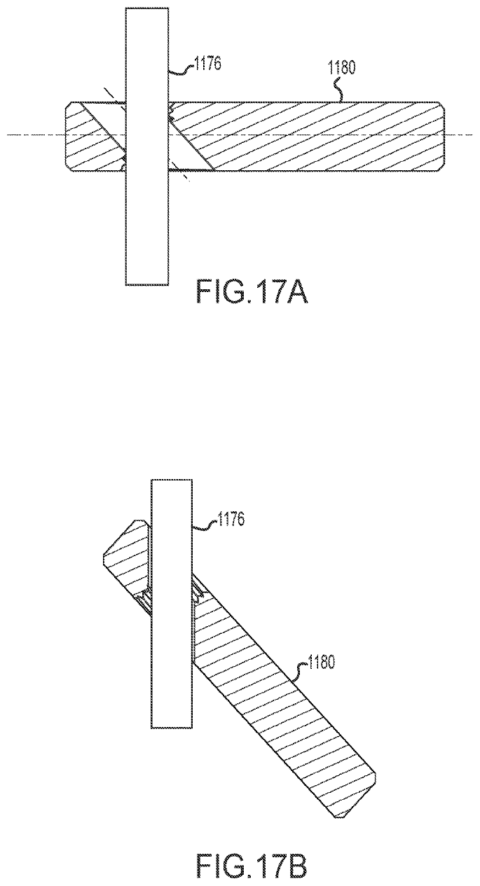

Turning to FIGS. 17A and 17B, a cross-section of the clamp arm 1180 in a first position and a second position are respectively shown. During use, the threaded shaft 1176 extends through the aperture 1182. When the clamp arm 1180 is in the first position, shown in FIG. 17A, the threaded bore 1192 is engaged with the threaded shaft 1176, thereby securing the clamp arm 1180 to the threaded shaft 1176. When the clamp arm 1180 is moved to the second position, shown in FIG. 17B, the threaded bore 1192 is no longer engaged with the threaded shaft 1176. In such position, the non-threaded bore 1186 may enable the clamp arm 1180 to be moved along a length of the threaded shaft 1176 until the clamp arm 1180 is moved to the first position. Such movement from the first position to the second position and vice versa is simply achieved by tilting or moving the clamp arm 1180 down towards the threaded shaft 1176 or moving the clamp arm 1180 to a perpendicular position with the threaded shaft 1176. Thus, the unique profile of the inner surface 1184 enables the clamp arm 1180 to be quickly and easily adjusted in a lateral direction.

During installation, the base 1108, 1110, 1112 of each clamp assembly 1102, 1104, 1106 may be secured to the corresponding protrusion 118A, 118B, 118C based on dimensions of the intensifier. A height of each spacer 1126, 1128, 1130 may be adjust whether by rotation of the knob 1174, or use of the clamp arm 1180 having the aperture 1182. A longitudinal direction of each spacer 1126, 1128, 1130 may also be adjusted. When the adaptor 1100 is ready to be installed on the intensifier 106, the first clamp assembly 1102 may be pivoted away from the mounting ring 110. The intensifier 106 may then be positioned between the mounting ring 110 and the spacers 1126, 1128, 1130. When the intensifier 106 is in place, the first clamp assembly 1102 may be pivoted back towards the mounting ring 110 until the spacer 1126 contacts and presses against the intensifier 106 so as to lock the adaptor 1100 to the intensifier 106. It will be appreciated that in other embodiments, the adaptor 1100 may be secured to the intensifier 106 with greater or less than three clamp assemblies. Though not shown, the adaptor 1100 is secured or fixed to the intensifier 106 at a plurality of contact points.

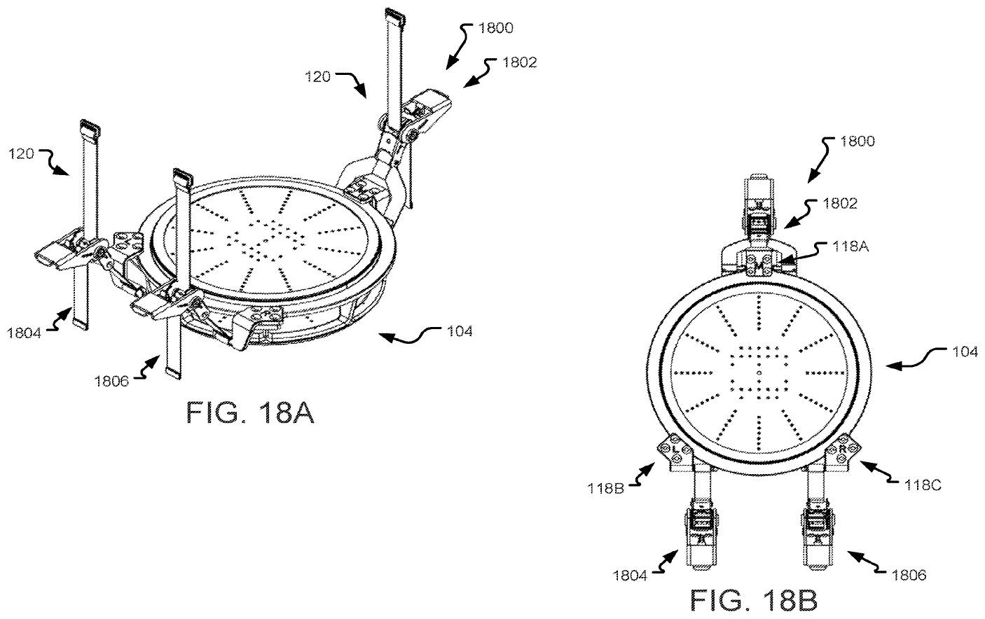

With reference to FIGS. 18A to 22, an adaptor 1800 according to at least one embodiment of the present disclosure is provided.

FIGS. 18A and 18B show a perspective view and a top view, respectively, of the adaptor 1800 fixed to the corrector assembly 104. The adaptor 1800 comprises the plurality of holders 120. In the illustrated embodiment, the plurality of holders 120 comprises a first belt assembly 1802, a second belt assembly 1804, and a third belt assembly 1804. It will be appreciated that in other embodiments the plurality of holders 120 comprises less or greater than three belt assemblies. As shown in FIG. 18B, the first belt assembly 1802 is fixed to the first protrusion 118A, the second belt assembly 1804 is fixed to the second protrusion 118B, and the third belt assembly 1806 is fixed to the third protrusion 118C. In other embodiments, the first belt assembly 1802, the second belt assembly 1804, and/or the third belt assembly 1806 may be fixed to any protrusion 118A, 118B, 118C.

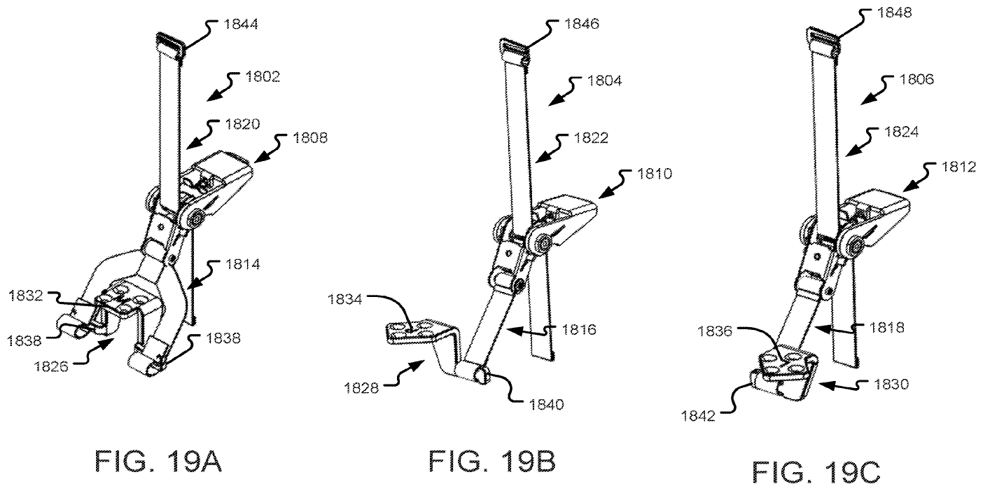

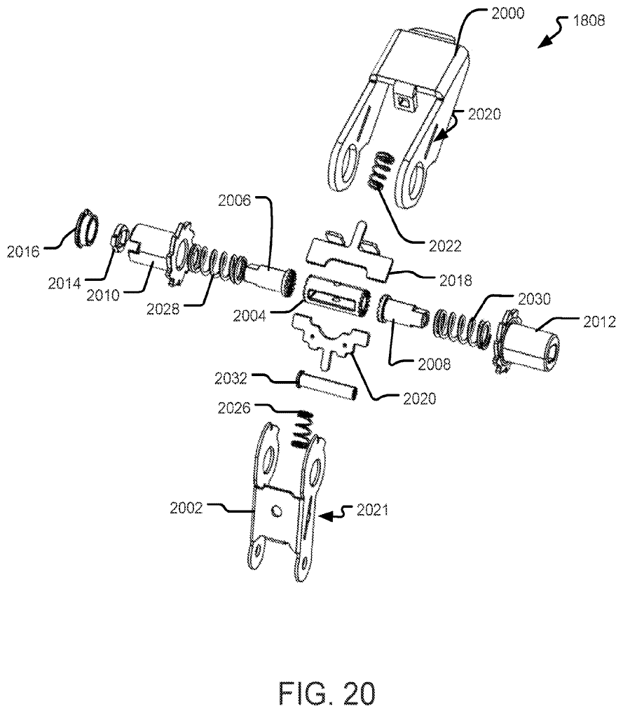

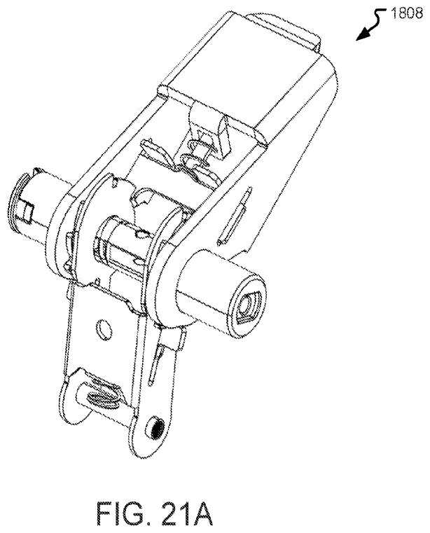

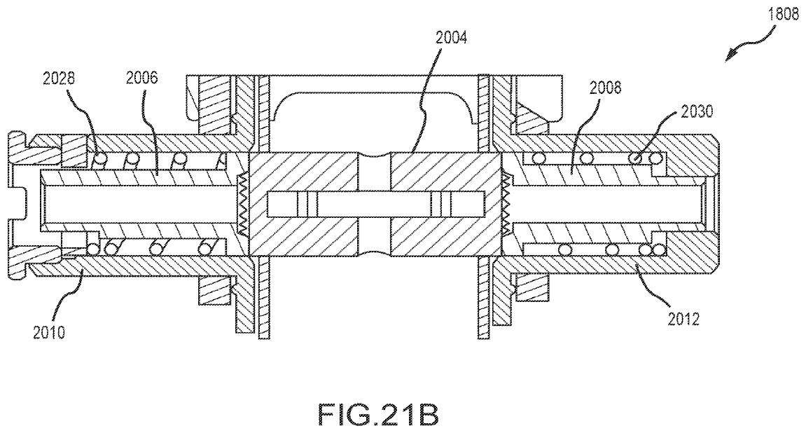

Turning to FIGS. 19A-19C, a perspective view of each of the first belt assembly 1802, the second belt assembly 1804, and the third belt assembly 1806 are respectively shown. Each belt assembly 1802, 1804, 1806 includes a ratchet 1808, 1810, 1812, a first belt 1814, 1816, 1818, a second belt 1820, 1822, 1824, and a belt base 1826, 1828, 1830. The first belt 1814, 1816, 1818 and the second belt 1820, 1822, 1824 may be formed from any fabric such as, for example, nylon, webbing, or the like. The belt base 1826, 1828, 1830 may be formed from any solid material such as, for example, aluminum, aluminum alloy, steel, titanium, or the like. The ratchet 1808, 1810, 1812, in some embodiments, is the ratchet described in FIGS. 20A-21B. In other instances, the ratchet 1808, 1810, 1812 may be any commercially available ratchet. Each of the belt bases 1826, 1828, 1830 includes a plate 1832, 1834, 1836 fixable to the corresponding protrusion 118A, 118B, 118C and at least one belt arm 1838, 1840, 1842 for receiving an end of a corresponding first belt 1814, 1816, 1818 to form a tie down anchor. In the illustrated embodiment, the at least one belt arm 1838 comprises two belt arms, the at least one belt arm 1840 and the at least one belt arm 1842 each comprise one belt arm. It will be appreciated that in other embodiments, the at least one belt arm 1838, 1840, 1842 may comprise one belt arm, two belt arms, or more than two belt arms.

The belt base 1826, 1828, 1830 is coupled to the ratchet 1808, 1810, 1812 by the first belt 1814, 1816, 1818. In other embodiments, the belt base 1826, 1828, 1830 may be coupled to the ratchet 1808, 1810, 1812 by any flexible member (e.g., a rope, a bungee, etc.) or any rigid member (e.g., a metal, a plastic, etc.). The second belt 1820, 1822, 1824 is received by the ratchet 1808, 1810, 1812 and couples to the intensifier 106 by, for example, a clip 1844, 1846, 1848. In other instances, the second belt 1820, 1822, 1824 may be coupled to the intensifier by, for examples, a loop end, a fastener, or the like. The clip 1844, 1846, 1848 may be received by a corresponding handle rail disposed on the imaging device 100.

Turning to FIG. 20, an exploded view of the ratchet 1808 is shown. Though the ratchet is referenced with respect to the ratchet 1808, it will be appreciated that the ratchet may be any of rachets 1808, 1810, 1812. The ratchet 1808 may be configured to limit an applied belt force to the imaging device 100 when each belt 1820, 1822, 1824 is tightened. The ratchet 1808 may include springs by which to adjust such applied belt force. The ratchet 1808 comprises a first handle 2000 coupled to a second handle 2002 by a central gear 2004 and a first side gear 2006 and a second side gear 2008. The first side gear 2006 and the second side gear extend through a first side ratchet 2010 and a second side ratchet 2012, respectively. A fix block 2014 and a side cover 2016 cover an end of the first side ratchet 2010. A first trigger 2018 is disposed in slot 2020 of the first handle 2000 and engages the first side ratchet 2010 and the second side ratchet 2012. A first spring 2022 biases the first trigger 2018 into engagement with the first side ratchet 2010 and the second side ratchet 2012. A second trigger 2024 is disposed in slots 2021 of the second handle 2002 and engages the first side ratchet 2010 and the second side ratchet 2012. A second spring 2026 biases the second trigger 2024 into engagement with the first side ratchet 2010 and the second side ratchet 2012. A third spring 2028 is positioned in the first side ratchet 2010 and a fourth spring 2030 is positioned in the second side ratchet 2012. A rivet 2032 is positioned at an end of the second handle 2002 and is configured to receive the first belt 1814, 1816, 1818 to couple the ratchet 1808 to the belt base 1826, 1828, 1830.

Turning to FIGS. 21A-21B, a perspective view and a cross-section view of the ratchet 1808 are respectively shown. During use, the third spring 2028 applies a bias force to the first side gear 2006 to bias the first side gear 2006 into the central gear 2004 and the fourth spring 2030 applies a bias force to the second side gear 2008 to bias the second side gear 2008 into the central gear 2004. The bias force may be based on the spring force of the third spring 2028 and the fourth spring 2030 selected. In other instances, the bias force may be adjusted by, for example, a tensioner. When a force applied to the first side gear 2006 and the second side gear 2008 by the first side ratchet 2010 and the second side ratchet 2012, respectively, is less than a threshold force, the first side gear 2006 and the second side gear 2008 are biased and engage the central gear 2004 and rotate the central gear 2004 (and, thereby ratcheting, for example, a belt engaged with the central gear 2004). When the force applied to the first side gear 2006 and the second side gear 2008 by the first side ratchet 2010 and the second side ratchet 2012, respectively, is greater than the threshold force, then the first side gear 2006 and the second side gear 2008 do not engage the central gear 2004 and thus, the central gear 2004 does not rotate. More specifically, in some embodiments the first side gear 2006, the second side gear 2008, and the central gear are each a crown gear. When the force applied to the first side gear 2006 and the second side gear 2008 is greater than a threshold force and the bias force, teeth of each of the first side gear 2006 and the second side gear 2008 slip against teeth of the central gear 2004. Further, such force pushes the first side gear 2006 and the second side gear 2008 against their respective bias forces, further allowing the first side gear 2006 and the second side gear 2008 to slip and rotate without engaging the central gear 2004. As such, the central gear 2004 does not rotate and a belt engaged with the central gear 2004 does not become tightened. Thus, for embodiments where the belt is tightened against an imaging device, the ratchet 1808 may prevent excess force from being applied to the imaging device.

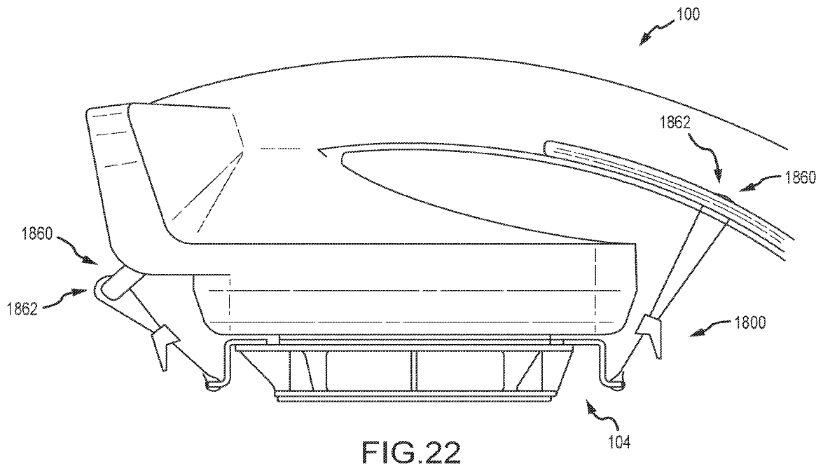

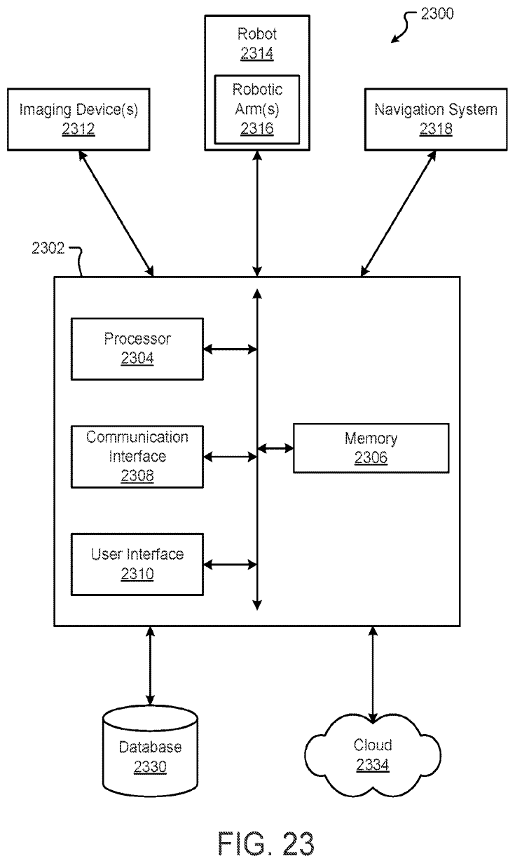

Turning to FIG. 22, the adaptor 1800 and the corrector assembly 104 are shown attached to the imaging device. The second belts 1820, 1822, 1824 are each attached to a plurality of contact points 1860. In the illustrated embodiment, the plurality of contact points 1860 are a plurality of handle rails 1862. During installation, one or more of the second belts 1820, 1822, 1824 may be extended so as to loosely connect the second belts 1820, 1822, 1824 to the corresponding handle rail 1862. Each second belt 1820, 1822, 1824 may then be tightened whether sequentially and/or simultaneously to draw the corrector assembly 104 to the intensifier 106 and then to hold and lock the corrector assembly 104 against the intensifier 106.

It will be appreciated that though the adaptors 300, 700, 1100, 1800 are described as attaching to an intensifier, that the adaptors 300, 700, 1100, 1800 may be attached or fixed to any component that may or may not be described herein.

Turning to FIG. 2300, a block diagram of a system 2300 according to at least one embodiment of the present disclosure is shown. The system 2300 may be used to orient and/or install an imaging device and/or carry out one or more other aspects of one or more of the methods disclosed herein. The system 2300 comprises a computing device 2302, one or more imaging devices 2312, a robot 2314, a navigation system 2318, a database 2330, and/or a cloud or other network 2334. It will be appreciated that the imaging devices 2312 may be the same as or similar to the imaging device 100 describe above with respect to FIGS. 1-22. Systems according to other embodiments of the present disclosure may comprise more or fewer components than the system 2300. For example, the system 2300 may not include the imaging device 2312, the robot 2314, the navigation system 2318, one or more components of the computing device 2302, the database 2330, and/or the cloud 2334.

The computing device 2302 comprises a processor 2304, a memory 2306, a communication interface 2308, and a user interface 2310. Computing devices according to other embodiments of the present disclosure may comprise more or fewer components than the computing device 2302.

The processor 2304 of the computing device 2302 may be any processor described herein or any similar processor. The processor 2304 may be configured to execute instructions stored in the memory 2306, which instructions may cause the processor 2304 to carry out one or more computing steps utilizing or based on data received from the imaging device 2312, the robot 2314, the navigation system 2318, the database 2330, and/or the cloud 2334.

The memory 2306 may be or comprise RAM, DRAM, SDRAM, other solid-state memory, any memory described herein, or any other tangible, non-transitory memory for storing computer-readable data and/or instructions. The memory 2306 may store information or data useful for completing, for example, any step of the methods 2400 described herein, or of any other methods. The memory 2306 may store, for example, one or more algorithms. Such algorithms may, in some embodiments, be organized into one or more applications, modules, packages, layers, or engines. Alternatively or additionally, the memory 2306 may store other types of data (e.g., machine learning models, artificial neural networks, etc.) that can be processed by the processor 2304 to carry out the various method and features described herein. Thus, although various components of memory 2306 are described as instructions, it should be appreciated that functionality described herein can be achieved through use of instructions, algorithms, and/or machine learning models. The data, algorithms, and/or instructions may cause the processor 2304 to manipulate data stored in the memory 2306 and/or received from or via the imaging device 2312, the robot 2314, the database 2330, and/or the cloud 2334.

The computing device 2302 may also comprise a communication interface 2308. The communication interface 2308 may be used for receiving image data or other information from an external source (such as the imaging device 2312, the robot 2314, the navigation system 2318, the database 2330, the cloud 2334, and/or any other system or component not part of the system 2300), and/or for transmitting instructions, images, or other information to an external system or device (e.g., another computing device 2302, the imaging device 2312, the robot 2314, the navigation system 2318, the database 2330, the cloud 2334, and/or any other system or component not part of the system 2300). The communication interface 2308 may comprise one or more wired interfaces (e.g., a USB port, an ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 2308 may be useful for enabling the device 2302 to communicate with one or more other processors 2304 or computing devices 2302, whether to reduce the time needed to accomplish a computing-intensive task or for any other reason.

The computing device 2302 may also comprise one or more user interfaces 2310. The user interface 2310 may be or comprise a keyboard, mouse, trackball, monitor, television, screen, touchscreen, and/or any other device for receiving information from a user and/or for providing information to a user. The user interface 2310 may be used, for example, to receive a user selection or other user input regarding any step of any method described herein. Notwithstanding the foregoing, any required input for any step of any method described herein may be generated automatically by the system 2300 (e.g., by the processor 2304 or another component of the system 2300) or received by the system 2300 from a source external to the system 2300. In some embodiments, the user interface 2310 may be useful to allow a surgeon or other user to modify instructions to be executed by the processor 2304 according to one or more embodiments of the present disclosure, and/or to modify or adjust a setting of other information displayed on the user interface 2310 or corresponding thereto.

Although the user interface 2310 is shown as part of the computing device 2302, in some embodiments, the computing device 2302 may utilize a user interface 2310 that is housed separately from one or more remaining components of the computing device 2302. In some embodiments, the user interface 2310 may be located proximate one or more other components of the computing device 2302, while in other embodiments, the user interface 2310 may be located remotely from one or more other components of the computer device 2302.

The imaging device 2312 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 2312, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 2312 may be used to obtain first image data (e.g., a first image) at a first time, and a second imaging device 2312 may be used to obtain second image data (e.g., a second image) at a second time after the first time. The imaging device 2312 may be capable of taking a 2D image or a 3D image to yield the image data. The imaging device 2312 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 2312 suitable for obtaining images of an anatomical feature of a patient. The imaging device 2312 may be contained entirely within a single housing, or may comprise a transmitter/emitter and a receiver/detector that are in separate housings or are otherwise physically separated.

In some embodiments, the imaging device 2312 may comprise more than one imaging device 2312. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 2312 may be operable to generate a stream of image data. For example, the imaging device 2312 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 2314 may be any surgical robot or surgical robotic system. The robot 2314 may be or comprise, for example, the Mazor X™ Stealth Edition robotic guidance system. The robot 2314 may be configured to position the imaging device 2312 at one or more precise position(s) and orientation(s), and/or to return the imaging device 2312 to the same position(s) and orientation(s) at a later point in time. The robot 2314 may additionally or alternatively be configured to manipulate a surgical tool (whether based on guidance from the navigation system 2318 or not) to accomplish or to assist with a surgical task. In some embodiments, the robot 2314 may be configured to hold and/or manipulate an anatomical element during or in connection with a surgical procedure. The robot 2314 may comprise one or more robotic arms 2316. In some embodiments, the robotic arm 2316 may comprise a first robotic arm and a second robotic arm, though the robot 2314 may comprise more than two robotic arms. In some embodiments, one or more of the robotic arms 2316 may be used to hold and/or maneuver the imaging device 2312. In embodiments where the imaging device 2312 comprises two or more physically separate components (e.g., a transmitter and receiver), one robotic arm 2316 may hold one such component, and another robotic arm 2316 may hold another such component. Each robotic arm 2316 may be positionable independently of the other robotic arm. The robotic arms may be controlled in a single, shared coordinate space, or in separate coordinate spaces.

The robot 2314, together with the robotic arm 2316, may have, for example, one, two, three, four, five, six, seven, or more degrees of freedom. Further, the robotic arm 2316 may be positioned or positionable in any pose, plane, and/or focal point. The pose includes a position and an orientation. As a result, an imaging device 2312, surgical tool, or other object held by the robot 2314 (or, more specifically, by the robotic arm 2316) may be precisely positionable in one or more needed and specific positions and orientations.

The robotic arm(s) 2316 may comprise one or more sensors that enable the processor 2304 (or a processor of the robot 2314) to determine a precise pose in space of the robotic arm (as well as any object or element held by or secured to the robotic arm).

In some embodiments, reference markers (i.e., navigation markers) may be placed on the robot 2314 (including, e.g., on the robotic arm 2316), the imaging device 2312, or any other object in the surgical space. The reference markers may be tracked by the navigation system 2318, and the results of the tracking may be used by the robot 2314 and/or by an operator of the system 2300 or any component thereof. In some embodiments, the navigation system 2318 can be used to track other components of the system (e.g., imaging device 2312) and the system can operate without the use of the robot 2314 (e.g., with the surgeon manually manipulating the imaging device 2312 and/or one or more surgical tools, based on information and/or instructions generated by the navigation system 2318, for example).

The navigation system 2318 may provide navigation for a surgeon and/or a surgical robot during an operation. The navigation system 2318 may be any now-known or future-developed navigation system, including, for example, the Medtronic StealthStation™ S8 surgical navigation system or any successor thereof. The navigation system 2318 may include one or more cameras or other sensor(s) for tracking one or more reference markers, navigated trackers, or other objects within the operating room or other room in which some or all of the system 2300 is located. The one or more cameras may be optical cameras, infrared cameras, or other cameras. In some embodiments, the navigation system may comprise one or more electromagnetic sensors. In various embodiments, the navigation system 2318 may be used to track a position and orientation (i.e., pose) of the imaging device 2312, the robot 2314 and/or robotic arm 2316, and/or one or more surgical tools (or, more particularly, to track a pose of a navigated tracker attached, directly or indirectly, in fixed relation to the one or more of the foregoing). The navigation system 2318 may include a display for displaying one or more images from an external source (e.g., the computing device 2302, imaging device 2312, or other source) or for displaying an image and/or video stream from the one or more cameras or other sensors of the navigation system 2318. In some embodiments, the system 2300 can operate without the use of the navigation system 2318. The navigation system 2318 may be configured to provide guidance to a surgeon or other user of the system 2300 or a component thereof, to the robot 2314, or to any other element of the system 2300 regarding, for example, a pose of one or more anatomical elements, whether or not a tool is in the proper trajectory, and/or how to move a tool into the proper trajectory to carry out a surgical task according to a preoperative or other surgical plan.

The database 2330 may store one or more images useful in connection with a surgery to be completed by or with the assistance of one or more other components of the system 2300, and/or any other useful information. The database 2330 may be configured to provide any such information to the computing device 2302 or to any other device of the system 2300 or external to the system 2300, whether directly or via the cloud 2334. In some embodiments, the database 2330 may be or comprise part of a hospital image storage system, such as a picture archiving and communication system (PACS), a health information system (HIS), and/or another system for collecting, storing, managing, and/or transmitting electronic medical records including image data.

The cloud 2334 may be or represent the Internet or any other wide area network. The computing device 2302 may be connected to the cloud 2334 via the communication interface 2308, using a wired connection, a wireless connection, or both. In some embodiments, the computing device 2302 may communicate with the database 2330 and/or an external device (e.g., a computing device) via the cloud 2334.

The system 2300 or similar systems may be used, for example, to carry out one or more aspects of any of the methods 2400 described herein. The system 2300 or similar systems may also be used for other purposes.

FIG. 2400 depicts a method 2400 that may be used, for example, for installing an adaptor to an imaging device.

The method 2400 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 2400. The at least one processor may perform the method 2400 by executing instructions stored in a memory such as the memory 106. The instructions may correspond to one or more steps of the method 2400 described below. The instructions may cause the processor to execute one or more algorithms.

The method 2400 comprises aligning an adaptor with an imaging device (step 2404). The adaptor may be the same as or similar to the adaptor 300, 700, 1100, 1800. The imaging device may be the same as or similar to the imaging device 100, 2312. The adaptor may connect a corrector assembly such as the corrector assembly 104 to an intensifier such as the intensifier 106. The adaptor may comprise a plurality of holders such as the plurality of holders 120 which are configured to secure the adaptor to the imaging device.

The imaging device may be any imaging device such as, for example an X-ray imaging device. The X-ray imaging device may comprise a source and a detector. In some embodiments, the source and the detector are supported by a C-arm at opposite ends of the C-arm. In other embodiments, the source may be supported by a first robotic arm and the detector may be supported by a second robotic arm.

The method 2400 also comprises adjusting the plurality of holders in one or more directions (step 2408). In some embodiments, the plurality of holders may comprise a clamping bar such as the clamping bar 302 and at least one side clamping bar such as the at least one side clamping bar 306. The clamping bar and the at least one side clamping bar are configured to contact the intensifier at a plurality of contact points. In other embodiments, the plurality of holders may comprise a bracket such as the bracket 702 and at least one side bracket such as the at least one side bracket 704. In such embodiments, the bracket and the at least one side bracket support spacers such as the spacers 722, 724, 726, 728. The spacers are configured to contact the intensifier at a plurality of contact points.

In still other embodiments, the plurality of holders may comprise a first clamp assembly such as the first clamp assembly 1102, a second clamp assembly such as the second clamp assembly 1104, and a third clamp assembly such as the third clamp assembly 1106. In such embodiments, the first clamp assembly, the second clamp assembly, and the third clamp assembly support spacers such as the spacers 1126, 1128, 1130. The spacers are configured to contact the intensifier at a plurality of contact points. In still other embodiments, the plurality of holders may comprise a first belt assembly such as the first belt assembly 1802, a second belt assembly such as the second belt assembly 1804, and a third belt assembly such as the third belt assembly 1806. In such embodiments, the first belt assembly, the second belt assembly, and the third belt assembly each comprise a clip 1844, 1846, 1848 which are attached or coupled to the plurality of contact points.

The method 2400 also comprises securing the plurality of holders at the plurality of contact points (step 2412). In some embodiments, securing the plurality of holders comprises tightening straps such as the straps 332 to compress the clamping bar and the at least one side bar against the intensifier at the plurality of contact points. In other embodiments, securing the plurality of holders comprises tightening the spacers against the intensifier at the plurality of contact points. In still other embodiments, securing the plurality of holders comprises ratcheting belts such as the belts 1820, 1822, 1824 to tighten the clip against the imaging device.

The present disclosure encompasses embodiments of the method 2400 that comprise more or fewer steps than those described above, and/or one or more steps that are different than the steps described above.

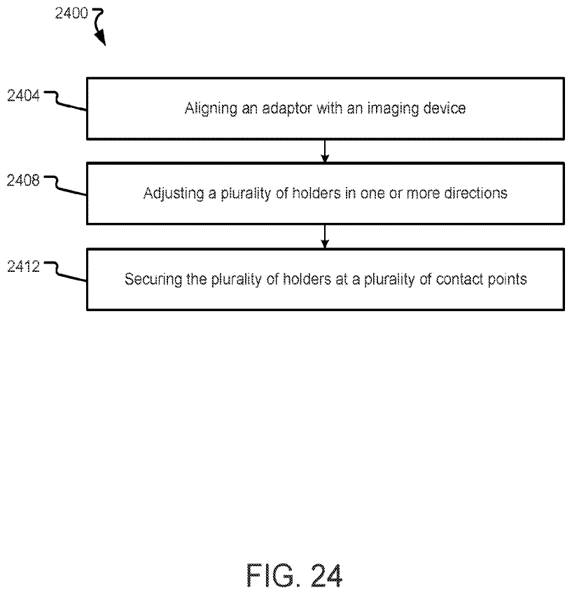

As noted above, the present disclosure encompasses methods with fewer than all of the steps identified in FIG. 24 (and the corresponding description of the method 2400), as well as methods that include additional steps beyond those identified in FIG. 2400 (and the corresponding description of the method 2400). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An adaptor assembly comprising:
a first arm, a second arm, and a third arm each comprising a first link pivotably connected to a base and a second link pivotably connected to the first link;
a clamping bar supported by the first arm and pivotably connected to the first link of the first arm;
a first side bar supported by the second arm and pivotably connected to the first link of the second arm; and
a second side bar supported by the third arm and pivotably connected to the first link of the second arm,
wherein the clamping bar, the first side bar, and the second side bar are securable to an imaging device.

2. The adaptor of claim 1, further comprising a first stopper configured to lock a corresponding first link and a second stopper configured to lock a corresponding second link.

3. The adaptor of claim 1, further comprising a grip disposed on each of the clamping bar, the first side bar, and the second side bar.

4. The adaptor of claim 1, wherein the first side bar and the second side bar are each coupled to the clamping bar via a corresponding strap, wherein each strap is adjustable by a corresponding adjuster.

5. The adaptor of claim 4, wherein the clamping bar, the first side bar, and the second side bar contact an imaging device at a plurality of contact points, and wherein each strap is adjusted to tighten and clamp each of the clamping bar, the first side bar, and the second side bar to the imaging device.

6. An adaptor assembly comprising:
a bracket comprising a wall extending perpendicularly from a base, a spacer laterally adjustable on the wall, and an adjustor configured to pivotably adjust the spacer; and
at least one side bracket, each side bracket comprising a wall extending perpendicularly from a base and a spacer laterally adjustable on the wall,
wherein the spacer of the bracket and each spacer of the at least one side bracket are configured to contact an imaging device at a plurality of contact points.

7. The adaptor of claim 6, wherein the wall of each of the at least one side bracket comprises a plurality of apertures spaced along a length of the wall, wherein the plurality of apertures are configured to receive the spacer, wherein the spacer is laterally adjustable by removing the spacer from a first set of apertures and securing the spacer to a second set of apertures.

8. The adaptor of claim 6, wherein the adjustor comprises a trigger and a protrusion each coupled to an adjustor base, wherein the protrusion is received in at least one of a first aperture and a second aperture spaced from the first aperture, wherein actuation of the trigger moves the protrusion from at least one of the first aperture and the second aperture, and wherein the adjustor base is pivotable when the trigger is actuated.

9. The adaptor of claim 8, wherein the trigger is biased away from the adjustor base.

10. The adaptor of claim 6, wherein the at least one side bracket comprises a first side bracket, a second side bracket, and a third side bracket.

11. An adaptor assembly comprising:
a first clamp assembly;
a second clamp assembly; and
a third clamp assembly,
wherein each of the first clamp assembly, the second clamp assembly, and the third clamp assembly comprises a clamp bracket extending from a clamp base, a clamp arm supported by the clamp bracket, and a spacer supported by the clamp arm, the spacer adjustable laterally, longitudinally, and rotationally.

12. The adaptor of claim 11, wherein the first clamp assembly also comprises an adjustor for pivotably adjusting the spacer.

13. The adaptor of claim 11, wherein the clamp arm of each assembly comprises a slot through which a threaded portion of the spacer extends through, wherein the spacer is slidable along the slot and securable to the clamp arm by a nut threading to the threaded portion.

14. The adaptor of claim 13, wherein each assembly comprises a lateral adjustor comprising a knob and a threaded shaft extending from the knob, wherein the clamp arm includes a threaded aperture configured to receive the threaded shaft, and wherein rotation of the knob in a first direction moves the clamp arm in a first lateral direction and rotation of the knob in a second direction moves the clamp arm in a second lateral direction opposite the first lateral direction.

15. The adaptor of claim 11, wherein the spacer of each assembly comprises a shaft and a contact point coupled to an end of the shaft in a ball and joint configuration.

16. The adaptor of claim 11, wherein each assembly comprises a lateral adjustor comprising an aperture disposed on the clamp arm, wherein the aperture comprises an inner surface having a threaded bore and a non-threaded bore, wherein an axis of the threaded bore is perpendicular to a center line of the clamp arm and an axis of the non-threaded bore is oblique to the center line.

17. The adaptor of claim 16, wherein the lateral adjustor further comprises a threaded shaft extending through the aperture, wherein when the clamp arm is in a first position, the threaded bore is engaged with the threaded shaft and when the clamp arm is in a second position, the threaded bore is disposed in the non-threaded bore.

18. An adaptor assembly comprising:
a first belt assembly;
a second belt assembly; and
a third belt assembly,
wherein each of the first belt assembly, the second belt assembly, and the third belt assembly comprises a first belt coupled to a belt base at one end and a ratchet at another end, a second belt coupled to the ratchet and a contact point of a plurality of contact points,
wherein the ratchet is configured to tighten the second belt,
wherein the second belt of each of the first belt assembly, second belt assembly, and the third belt assembly attach to a first contact point, a second contact point, and a third contact point,
wherein the first contact point is opposite the second contact point and the third contact point.

19. The adaptor of claim 18, wherein the plurality of contact points comprises a plurality of handle rails.

20. The adaptor of claim 18, wherein the belt base of each assembly comprises a plate and at least one belt arm, wherein the plate is configured to secure the assembly to a corrector assembly and the at least one belt arm configured to receive the first belt.

21. The adaptor of claim 18, wherein the ratchet comprises a central gear configured to engage with a first side gear and a second side gear, wherein the first side gear and the second side gear are configured to rotate the central gear when the first side gear and the second side gear are rotating and are engaged with the central gear.

22. The adaptor of claim 21, wherein the first side gear and the second side gear are configured to disengage the central gear when a force applied to the first side gear and the second side gear exceed a threshold force.

23. The adaptor of claim 22, wherein the first side gear and the second side gear are biased to the central gear by a biasing force, and wherein the first side gear and the second side gear are configured to disengage the central gear when the force applied is greater than the biasing force and the threshold force.

24. The adaptor of claim 23, wherein each of the first side gear, the second side gear, and the central gear comprise a crown gear.

25. The adaptor of claim 23, wherein the ratchet comprises at least one spring configured to apply the biasing force.

26. An adaptor assembly comprising:
a plurality of spacers configured to contact a plurality of contact points; and
a plurality of holders configured to be adjustable in one or more directions and to support the plurality of spacers.

27. The adaptor of claim 26, wherein the plurality of holders comprises a bracket comprising a wall extending perpendicularly from a base, one spacer of the plurality of spacers laterally adjustable on the wall, and an adjustor configured to pivotably adjust the spacer, and wherein the plurality of holders further comprises at least one side bracket, each side bracket comprising a wall extending perpendicularly from a base and a corresponding spacer of the plurality of spacers laterally adjustable on the wall.

28. The adaptor of claim 26, further comprising a first arm, a second arm, and a third arm, each comprising a first link pivotably connected to a base and a second link pivotably connected to the first link; and wherein the plurality of holders comprises a clamping bar supported by the first arm and pivotably connected to the first link of the first arm, a first side bar supported by the second arm and pivotably connected to the first link of the second arm, and a second side bar supported by the third arm and pivotably connected to the first link of the second arm.

29. The adaptor of claim 26, wherein the plurality of holders comprises a first clamp assembly; a second clamp assembly; and a third clamp assembly, wherein each of the first clamp assembly, the second clamp assembly, and the third clamp assembly comprises a clamp bracket extending from a clamp base, a clamp arm supported by the clamp bracket, and a spacer supported by the clamp arm, the spacer adjustable laterally, longitudinally, and rotationally.

30. The adaptor of claim 26, wherein the plurality of holders comprises a first belt assembly, a second belt assembly, and a third belt assembly, wherein each of the first belt assembly, the second belt assembly, and the third belt assembly comprises a first belt coupled to a belt base at one end and a ratchet at another end, a second belt coupled to the ratchet and a contact point of a plurality of contact points, and wherein the ratchet is configured to tighten the second belt.
